(19) United States
(12) Patent Application Publication
(10) Pub. No.: US 2002/0169264 A1
(43) Pub. Date: Nov. 14, 2002

(54) Polymers incorporating peptides

(75) Inventors: David C. Jackson, N. Balwyn (AU); Neil M. O'Brien-Simpson, Brunswich (AU); Lorena E. Brown, North Balwyn (AU); Nicholas J. Ede, East Malvern (AU); Evelyn R. Brandt, Windsor (AU); Michael F. Good, The Gap (AU); Weiguang Zeng, North Melbourne (AU)

Correspondence Address: Thomas M. Boyce, Esq., FULBRIGHT & JAWORSKI L.L.P., Suite 2400, 600 Congress Avenue, Austin, TX, 78701, US

(73) Assignee: The Council of the Queensland Institute of Medical Research

(21) Appl. No.: 10/044,034

(22) Filed: Jan. 11, 2002

Related U.S. Application Data
(62) Division of application No. 08/944,147, filed on Oct. 6, 1997.

(30) Foreign Application Priority Data
Feb. 11, 1997 (AU) PO 5071

(51) Int. Cl. G01N 33/53; C08H 1/00

(52) U.S. Cl. 526/238.1; 435/7.1

(57) ABSTRACT

The present invention provides polymers incorporating peptides. The polymers comprise polymarised units of (1) CH₂═CR₄—CO—X—R₁ and (2) CH₂═CR₃—CO—R₂ and optionally one or more other monomers, in which R₁ is a peptide. Each R1 may be the same, or is preferably different. In another embodiment the present invention provides polymers formed from CH₂═CR₄—CO—X—R₁ and optionally one or more other monomers, in which X is a spacer having a length equivalent to 1 to 30 single C—C bonds and R₁ is a peptide, each R₁ being the same or different. The invention further relates to methods producing the polymers and methods of inducing an immune response using the polmers.

The present invention relates to polymers incorporating peptides. The polymers may be used for raising an immune response or in delivery of the peptides or as a diagnostic tool.

Synthetic peptides are widely used to generate site-specific antibodies, a fact which has stimulated considerable interest in evaluating their use as vaccine candidates. The advantages of this approach include safety, as there is no requirement for infectious material, and the ability to chemically define the product Although there are a number of potential advantages in developing synthetic peptide-based vaccines, there are currently major problems limiting their exploitation. Many of these limitations center around the small size, low copy number and low immunogenicity of peptide-based immunogens These problems have traditionally been addressed by conjugating peptide epitopes to carrier proteins, a procedure which provides T-cell help for synthetic peptide B-cell epitopes and provides multivalent display of epitopes to the immune system. While this method usually elicits some antibody directed to the peptide as well as to the carrier, prior exposure of an individual to the carrier protein can result in suppression of the B-cell response to the peptide^{1,2} and the coupling procedure can also have deleterious effects on the integrity of the peptide determinants)³.

A total synthetic approach in which peptide epitopes are incorporated in tandem into a linear sequence⁴⁻⁸ has also been described. This approach is limited by the length of the immunogen that can be synthesised and also by the possibility of introducing novel immunogenic and irrelevant sequences at the junction of otherwise distinct determinants. To overcome these problems Tam and collegues⁹⁻¹¹ have assembled multiple peptides onto a branched oligolysine support, and the present inventors and others^{12, 13} have synthesised peptides on cross-linked acrylamide resins in which the cross-links are cleaved on exposure to trifluoroacetic acid resulting in a long single chain polyamide to which multiple copies of the peptide are attached.

With each of these synthetic approaches there are, however, limitations to the degree of purity of the component peptides that can be achieved. The problem of purity of oligomeric synthetic peptide-based vaccines has been addressed in a number of studies including the elegant chemical ligation approaches described by Rose¹⁴ and Tam^{15,16}. These approaches also offer greater flexibility by permitting, in principle, the conjugation of different purified peptides onto a template support although the number of different peptide epitopes that can be incorporated into these structures is still restricted. Because T- and B-cell epitopes defined in a single host of a particular MHC type may be inadequate for eliciting immunity in outbred populations and because many diseases are caused by organisms where the target antigens are polymorphic, this restriction in the number of different epitopes that can be incorporated is an important consideration.

As further background to the present invention, reference may also be made to an article by Birr et al. entitled “Anti-FeLV Synthetic Peptide Vaccine Development” in PEPTIDES—Chemistry and Biology; Proceedings of the Twelfth American Peptide Symposium, Jun. 16-21, 1991 at Cambridge, Mass., U.S.A. (edited by John A. Smith and Jean E. Rivier) which discussed construction of synthetic vaccines involving a polymeric vaccine containing the seven best FeLV epitopes which involved N-terminal acryloylation of each epitope and polyamide copolymerisation of all seven epitopes into a macromolecular but water soluble “brush”-type antigen. The epitopes were identified in Nick et al., 1981, Proc. Acad. Sci. U.S.A. 78 3824. However, the specific conditions of polymerisation which took place in relation to formation of the synthetic polymeric vaccine were not described and thus the disclosure in Birr et al. could not constitute an enabling disclosure in regard to production of the vaccine.

The Birr et al. polymeric vaccine, as an immunogen, only had limited effectiveness in antibody response in providing specific antibody titres in vaccination trials which were intermediate between a higher titre obtained by a first vaccine derived from N-terminal conjugation of each of the same seven epitopes with N-palmitoyl-S-[2,3 bis-(palmitoyloxy)-(2RS)-propyl]-(R) cysteinyl-serine as discussed in Jung et. al., 1985, Int. Ed. Engl. 24 872 and a lower titre obtained by another vaccine which was produced by recombinant co-expression in E. coli as a C-terminal fusion product of β-galactosidase as discussed in Clarke et al., 1987, Nature 330 381.

It was also noted in the Birr et al. reference that the polymeric vaccine had substantially less than 30% effectiveness as a vaccine when compared to the first vaccine and such limited effectiveness would be commercially unacceptable.

It is believed that the apparent ineffectiveness of the polymeric vaccine reported in Birr et al. involving N-terminal acryloylation of each epitope and subsequent polyamide copolymerisation may have been a consequence of the fact that steric limitations which were applicable to the polymerisation process were not taken into account in the polymerisation process having regard to location of each peptide with the polymer.

Like the chemical ligation procedures, the approach developed by the present inventors is also “modular” and permits peptides to be synthesised, purified and then assembled into polymers. In this case, however, very large (>600,000 Dalton) molecular species can be assembled with virtually any number of the same or different epitopes. The technique depends upon acylation of an amino group at some point in the peptide sequence, while still attached to the solid phase support with side chain protecting groups intact, with the acryloyl (CH2═CH—CO—) group. The orientation of a peptide within a polymer may affect the resulting immune response as described, for example, in Silversides et al. (A synthetic luteinizing hormone releasing hormone vaccine. I. Conjugation and specificity trials in BALB/c mice. Silversides-D. W.; Allen-A. F.; Misra-V.; Qualtiere-L.; Mapletoft-R. J.; Murphy-B. D. J-Reprod-Immunol. 1988 August; 13 (3): 249-61). It may therefore be necessary in some cases to have the peptide epitope attached to the polymer backbone at its C-terminus or from some other point within the sequence rather than the N-terminus. The availability of lysine derivatives which have different types of amino protecting groups, for example, 9-fluorenylmethoxycarbonyl (Fmoc), Mtt (4-methyltrityl) and Dde (4,4-dimethyl-2,6-dioxocyclohex-1-ylidene ethyl), allows orthogonal chemistries to be used to selectively expose a particular amino group by removal of its protecting group such that the acryloyl group can be introduced at a specified point or points within the peptide sequence. Following cleavage, deprotection and purification, the peptides can be polymerised by free radical initiation of chain elongation in much the same way that acrylamide is routinely polymerised into polyacrylamide gels. In this way peptides are assembled into polymers in which the peptides form side chains pendant from an alkane backbone (FIG. 1). The method allows purification of the individual determinants, avoids errors inherent in long sequential syntheses and, in contrast to an approach described by Eckstein¹⁷, has the advantage that protected peptide fragments are not used thereby avoiding solubility and purification problems. Furthermore, multiple copies of many different peptide epitopes can be incorporated into a single polymeric structure to allow utilisation of the range of T cell epitopes required for outbred populations, in conjunction with epitopes representing different pathogenic serodemes. This approach may prove to be a significant advance in synthetic vaccine technology.

Accordingly in a first aspect the present invention consists in a polymer comprising polymerised units of (1) CH₂═CR₄—CO—X—R₁ and (2) CH₂═CR₃—CO—R₂, and optionally one or more other monomers, in which X is absent or is a spacer having a length equivalent to 1 to 30 single C—C bonds;

R₁ is a peptide, each R₁ being the same or different;

R₂ is NH2,

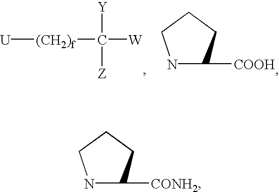

in which U is O or NH;

f is an integer from 0 to 17;

Y is H, COOH, CO—NH₂;

W is H, CH₃, CH₂CO—NH₂, CH₂COOH, CH₂OH, CH(CH₃)OH,

CH₂SH, CH₂CH₂COOH, CH₂CH₂CO—NH₂, CH₂CH(CH₃)₂,

CH(CH₃)CH₂CH₃, (CH₂)₄NH₂, CH₂CH₂SCH₃,

CH(CH₃)₂,

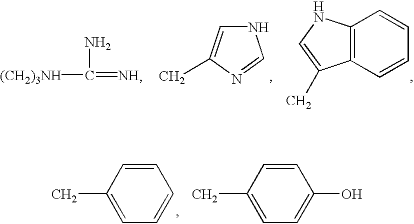

Z is H, (CH₂)ₕQT, in which h is 0-10, Q is O, NH, CH₂ and T is a lipid, labelling molecule, targeting molecule or functional group;

R₃ is CH₃, H, NH₂, OH, CN or halogen, each R₃, being the same or different;

R₄ is CH₃, H, NH₂, OH, CN or halogen, each R₄ being the same or different; and

the ratio of (1):(2) being in the range of about 1:1 to about 1:1000.

In a second aspect the present invention consists in a polymer formed from CH₂═CR₄—CO—X—R₁ and optionally one or more other monomers, in which X is a spacer having a length equivalent to 1 to 30 single C—C bonds;

R₁ is a peptide, each R₁ being the same or different;

R₄ is CH₃, H, NH₂, OH, CN or halogen, each R₄ being the same or different.

In a third aspect the present invention consists in a method of preparing a peptide polymer having a polymer backbone and a plurality of pendant peptides attached thereto, the method comprising the steps of:

(i) preparing monomers (1) CH₂═CR₄—CO—X—R₁, and (2) CH₂═CR₃—CO—R₂, and optionally one or more other monomers, in which X is absent or is a spacer having a length equivalent to 1 to 30 single C—C bonds;

R₁ is a peptide, each R₁, being the same or different;

R₂ is NH2,

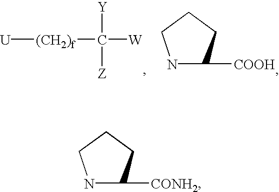

in which U is O or NH;

f is an integer from 0 to 17;

Y is H, COOH, CO—NH₂;

W is H, CH₃, CH₂CO—NH₂, CH₂COOH, CH₂OH, CH(CH₃)OH,

CH₂SH, CH₂CH₂COOH, CH₂CH₂CO—NH₂, CH₂CH(CH₃)₂,

CH(CH₃)CH₂CH₃, (CH₂)₄NH₂, CH₂CH₂SCH₃,

CH(CH₃)₂,

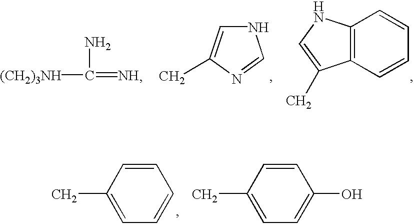

Z is H, (CH₂)ₕQT, in which h is 0-10, Q is O, NH, CH₂ and T is a lipid, labelling molecule, targeting molecule or functional group;

R₃ is CH₃, H, NH₂, OH, CN or halogen, each R₃ being the same or different;

R₄ is CH₃, H, NH₂, OH, CN or halogen, each R₄ being the same or different; and

(ii) polymerising the monomers in the presence of a free radical initiator.

In a fourth aspect the present invention consists in a method of raising an immune response in an animal to peptide epitopes, the method comprising administering to the animal a composition comprising an acceptable carrier and a polymer comprising polymerised units of (1) CH₂═CR₄—CO—X—R₁ and (2) CH₂═CR₃—CO—R₂, and optionally one or more other monomers, in which X is absent or is a spacer having a length equivalent to 1 to 30 single C—C bonds;

R₁ is a peptide, each R₁ being the same or different, at least one of R₁ being the peptide epitope;

R₂ is NH2,

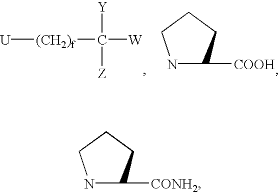

in which U is O or NH;

f is an integer from 0 to 17;

Y is H, COOH, CO—NH₂;

W is H, CH₃, CH₂CO—NH₂, CH₂COOH, CH₂OH, CH(CH₃)OH,

CH₂SH, CH₂CH₂COOH, CH₂CH₂CO—NH₂, CH₂CH(CH₃)₂,

CH(CH₃)CH₂CH₃, (CH₂)₄NH₂, CH₂CH₂SCH₃,

CH(CH₃)₂,

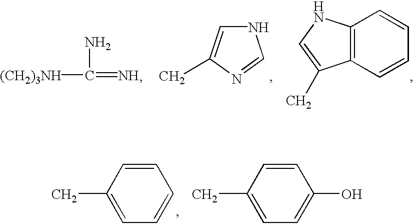

Z is H, (CH₂)ₕQT, in which h is 0-10, Q is O, NH, CH₂ and T is a lipid, labelling molecule, targeting molecule or functional group;

R₃ is CH₃, H, NH₂, OH, CN or halogen, each R₃ being the same or different;

R₄ is CH₃, H, NH₂, OH, CN or halogen, each R₄ being the same or different; and

the ratio of (1):(2) being in the range of about 1:1 to about 1:1000.

The polymer will typically be random, however, it may be block or alternating.

The polymer may also be cross-linked. This may be achieved any suitable cross-linking agent such as bisacrylamide. The cross-linking agent will be of the general formula CH₂═CH—CO—R₅—CO—CH═CH₂ in R₅ can be an amino acid sequence, NH(CH₂)₂SS(CH₂)₂NH or NH(CH₂)ₖNH in which k is 1 to 10.

As will be readily appreciated the polymer will normally include a plurality of R₁ groups. It is preferred that the peptides R₁ are epitopes and that this plurality of R₁ groups provides a mixture of T cell and/or B cell epitopes.

It will be recognised that the second of the elements in the polymer basically acts as a spacer between R₁ groups. This spacer may be acrylamide (R₂═NH₂), however it is preferred that the spacer is formed of acryloylated amino acids and in particular serine or glutamic acid. It will also be understood that differing spacers may be used in the polymer (varying R₂). In other embodiments R₂ may include a lipid component or may include a group which will direct the polymer to a particular target. For example R₂ may include lipids such as palmitic acids or Nα-Palmitoyl-S-[2,3-bis(palmitoyloxy)-(2RS)-propyl]-L-cysteine. As will be appreciated this is one way ill which self-adjuvanting properties may be added to the polymer. It is also possible to include molecules with labelling functions in R₂ e.g. fluorescamine, metal ion chelating molecules for chelating radioactive metal ions etc. Molecules with targeting properties may also be included in R₂. For example folic acid to target cancer cells. As will also be readily appreciated that molecules which modify the physiochemical properties, such as solubility, viscosity etc., of the polymer may be included in R₂.

In one aspect a spacer group X is present, however, in others others its presence is optional. It is presently preferred that the polymer includes this spacer so that the peptide R₁ is spaced away from the polymer backbone. It is preferred that spacer group X includes an enzymaticly cleavable site. This is particularly preferred where R₁ are peptide epitopes. Examples of cleavable sites include the peptides GLFG and VYLKY.

It is also preferred that the ratio (1):(2) is in the range 1:10 to 1:50.

While the bulk of the discussion and examples in this application relate to polymers in which R₁ is or are peptide epitopes it is to be understood that it is not essential that R₁ is or are peptide epitopes. The present invention contemplates polymers in which R₁ is or are other biologically active peptides, such as hormones etc.. For example the polymers of the present invention may provide useful vehicles for the delivery of a range of biologically active peptides. In this arrangement by adjusting the spacer X it may be possible to produce polymers which release the peptide over prolonged periods. It is also possible to cross-link a plurality of the polymers of the present invention by providing cross linkable moieties in R₂. This may be beneficial in providing delayed release of R₁.

As will be understood the invention provides a method of preparation of a polymeric immunogen having a polymer backbone and a plurality of pendant peptides attached thereto. In general the method includes the steps of:

(i) optionally adding a lateral spacer to each of the peptides;

(ii) acryloylating each of the peptides with an acryloylating agent; and

(iii) polymerising each of the acryloylated peptides, and optionally a linear spacer, in the presence of a free radical initiator to prepare the required polymeric immunogen wherein said linear spacer separates peptide arrays from each other along the polymer backbone and said lateral spacers space each peptide from the polymer backbone.

The peptides in step (i) may be obtained from any suitable source which may include extraction of a natural protein, chemical cleavage of a natural synthetic or recombinant protein, recombinant expression of an oligopeptide or from a naturally occurring small protein. However, preferably each peptide is synthesised either manually or by using an automatic peptide synthesiser.

The peptides in step (i) may be synthesised using solution synthesis or solid phase synthesis as described, for example, in Chapter 9 entitled “Peptide Synthesis” by Atherton and Sheppard which is included in a publication entitled “Synthetic Vaccines” edited by Nicholson and published by Blackwell Scientific Publications. Preferably a solid phase support is utilised which may be polystyrene gel beads wherein the polystyrene may be cross-linked with a small proportion of divinylbenzene (e.g. 1%) which is further swollen by lipophilic solvents such as dichloromethane or more polar solvents such as dimethylformamide (DMF). The polystyrene may be functionalised with chloromethyl or aminomethyl groups. Alternatively, cross-linked and functionalised polydimethyl-acrylamide gel is used which may be highly solvated and swollen by DMF and other polar aprotic solvents. Other supports can be utilised based on polyethylene glycol which is usually grafted or otherwise attached to the surface of inert polystyrene beads. In a preferred from, use may be made of a variety of commercial solid supports or resins such as PAL-PEG, PAK-PEG, KA, KR or TGR.

In solid phase synthesis, use is made of removable blocking groups which mask reactivity in the α-amino and side chain functional groups.

It will also be appreciated from the foregoing that each peptide may have additional moieties selected from carbohydrate, lipid, nucleotide or nucleoside as may be desired. Especially preferred are oligosaccharides, oligonucleotides or glycosidic groups.

In regard to step (ii), use may be made of an suitable acryloylating agent such as acryloyl chloride, cyanoacryloyl chloride, methacryloyl chloride or esters of acrylic acid or methacrylic acid.

After step (ii) the peptide may be cleaved from the resin and the protecting groups removed by a suitable cleaving agent or sequential use of cleavage reagents. Preferably the cleaving agent does not contain a thiol group because the thiol group may react with double bonds.

After removal of the resin, the peptides may be purified in any suitable manner such as, for example, by reverse phase, ion exchange or size exclusion chromatography.

In regard to step (iii) polymerisation of the acryloylated peptides may occur in any suitable manner. A solution of the acryloylated peptides may be treated with a reagent capable of generating a very small concentration of free radicals. In aqueous solution, a trace of hydrogen peroxide and a ferrous salt can be used or a trace of ammonium persulphate with N,N,N′,N′-tetramethylenediamine. The peptide entities are therefore pendant from a linear backbone. Each peptide could terminate in either a carboxyl or carboxyamide group to mimic the natural situation of the particular epitope, i.e. whether the particular epitope forms the C-terminus of the protein or whether it represents an internal sequence of amino acids.

In the case of the co-polymerisation of two or more acryloyl peptides, the distribution of the peptides along the polymer backbone may be controlled to some extent by controlling the stoichiometry of component peptides in the polymerisation mixture. Such an arrangement may even allow for the simulation of conformational determinants of the native protein where different peptides representing regions remote in sequence of the parent protein are attached to the backbone.

Co-polymerisation of the acryloyl peptides with different amounts of a suitably functionalised reagent such as N, N′-dimethylacrylamide or acryloylated amino acids, would allow for the pendant peptides to be separated to a greater or lesser extent from one another along the polymer backbone. This arrangement may prove necessary in some cases where steric hindrance or adventitious interactions between neighbouring peptides occurs. It could also increase or decrease solubility of the resulting polymeric peptide as desired.

In relation to the polymerisation of different peptides, this may occur by preparing the acryloyl derivative of a first peptide and the methacryloyl derivative of a second peptide and then co-polymerise the two. The sequence order of the two peptides along the backbone of the resultant polymer could well be different (and possibly more ordered) than might be the case for the co-polymerisation of the acryloylated derivatives of the first peptide and the second peptide where a more random arrangement could be expected.

Small amounts of low molecular weight acrylic amides or esters, chosen for specific features, could also be added to the mixture of monomers prior to the polymerisation step. Such additional monomers could include an acryloyl compound having a radioactive label attached, or an acryloyl derivative of a fluorescent or chemiluminescent reagent. The resulting polymer would then contain, in chemical side chains distinct from the peptide epitopes themselves, the potential for subsequent easy detection of that polymer. This feature would be particularly advantageous at the research and development stage.

Assembly of an appropriate combination of B- and T-cell epitopes should allow antibody and T-cell production in animals in a wide range of different histocompatibility types. This is of great importance in design of any vaccine to be administered to an outbred population such as man or his domestic animals. It is also possible that assembly of carefully chosen peptides and incorporation of other reagents such as lipid-containing material as linear or lateral spacers into the polymerisation mixture could obviate the need for adjuvants.

“Slow release” form of these peptide polymers may become available through preparation of the peptide acryloyl derivative and polymerisation in the presence of a cross-linking reagent such as bisacrylamide. These polymers may also express interesting biological activity per se by virtue of the fact that each polymer molecule would present a very high local concentration of the active monomeric unit. Numerous peptide hormones are now known including insulin, gastrin, oxytocin, vasopressin, adrenocorticotrophic hormone (ACTH), growth hormone, cholecystokinin, bombesin, substance P, relaxin, encephalin, angiotensin, somatostatin, bradykinin and so on. These molecules and many variants have been synthesised and tested, especially in a search for agonist and antagonist analogues and also for modified peptides with improved biological selectivity and stability. A “poly-vasopressin” or analogue, for example, might be extremely efficient in blocking the natural vasopressin receptor on a cell surface.

It might also be feasible to confine the hormonal or other pharmaceutical action to a particular site or organ of the experimental animal by virtue of the high molecular weight of the polymer, especially if the polymer has some cross-linking to render it insoluble.

Immobilisation of a biologically-active peptide on a polymeric support already has many known applications in research, for example in the isolation of receptor molecules, purification of antibodies and in immunochemical analysis. Cross-linked polyacrylamides with specific peptide side chains, such as those described here, could serve similar functions.

In order that the nature of the present invention may be more clearly understood preferred forms thereof will now be described with reference to the following non-limiting examples.

### FIGURE LEGENDS

FIG. 1. Scheme for the preparation of synthetic peptide based polymers. Peptides are assembled in the normal way on solid phase supports and then acylated at the N-terminus with acryloyl chloride. Following removal of the peptide from the support and concommitant removal of the side chain protecting groups, the peptide epitopes are purified and polymerised by exposure to free radical.

FIG. 2. Analytical reverse phase HPLC chromatograms of crude H₂N—P8 (A) and crude N-acryloyl P8 (B) using a Vydac C18 column (4.6 ×300 mm) installed in a Waters HPLC system. The chromatogram was developed at a flow rate of 1 mL min⁻¹ using a gradient, 0-100% solvent B developed over 30 mins.

FIG. 3; ¹H NMR of N-acryloyl-GFGA. The N-acryloylated peptide was cleaved from the resin using reagent B, purified by RP-HPLC and examined by ¹H NMR. The characteristic chemical shift for the acryloyl group, ¹H NMR (d6-DMSO) 6.27 (dd, 1H, Jₜᵣₐₙₛ=17.3 Hz, J_{Cis} =10.34 Hz CH═CH₂), 6.08 (dd, 1H, j=17.13 Hz, J_{gem}=1.8 Hz, H-trans), 5.57 (dd, 1H, J=11.16 Hz, J_{gem}=1.4 Hz, H-cis) are indicated.

FIG. 4: Gel permeation chromatography of monomeric and polymeric peptides. CH₂═CHCO—P6 (______) and NH₂—P6 (- - -) were subjected to the polymerisation protocol and introduced to a column (1×30 cm) of Superdex 200 and the chromatogram developed at a flow rate of 0.5 mL min⁻¹ in 50mM NH₄HCO₃. The column effluent was monitored at 280nm to detect tryptophan and tyrosine residues. The arrows indicate the retention time of the molecular weight standards; thyroglobulin 669 kDa (A), bovine serum albumin 67 kDa (B) and ribonuclease 13.7 kDa (C).

FIG. 5: Antigenic integrity of peptides and peptide polymers. The binding of anti-P8 antisera to polymerised P8 (▪), P8 monomer (□) or to polyacrylamide (Δ) and the binding of MAb 1/1 to polymerised P5 (●), P5 monomer (◯) or to polyacrylamide (∇) was examined by ELISA.

FIG. 6. Ability of soluble monomeric and polymeric peptides to inhibit antigen-antibody binding. Dilutions of peptide, or peptide polymer were mixed with a constant amount of antibody and added to wells of a microtiter tray coated with peptide antigen. Binding of the antibody was measured by ELISA and the result expressed as the percentage of the antibody bound in the absence of inhibitor. (A) P2 monomer (⋄), polymerised P2 (Δ) or polymerised P4 (◯) were incubated with MAb 1/1 and binding assessed on P5 coated wells. (B) P8 monomer (⋄), polymerised P8 (Δ) or polymerised P4 (●) were incubated with anti-P8 antisera and binding assessed on P8coated wells.

FIG. 7. Elution profile of poly (T+B1+B2) from a column (1×30cm) of Superose 6 installed in a Waters HPLC system. The chromatogram was developed in 50 mM ammonium bicarbonate at a flow rate of 0.5ml/min. Polymers eluted in the void volume of the column (ca. 7.5ml) and the monomeric forms of the peptides eluted in the position indicated by the arrow. The calibration curve for the column was constructed using the standard proteins shown.

FIG. 8. Effect of free radicals on the antigenicity of synthetic peptides. MAbs 1/1 and 2/1 were examined for their ability to bind to untreated peptide 306PKYVKQNTLKLATGMRNVPEKQT328 (●) and peptide exposed to various concentrations of ammonium persulphate. The molar ratios of ammonium persulphate to peptide were; 0.1:1, (⋄); 1:1, (∘); 10:1, (Δ); 100:1, (∇); and 1000:1, (z,900 ) The level of binding of NMS to each of the different peptide preparations (▪) is also shown.

FIG. 9. Antigenic integrity of polymeric peptides. Binding of MAb 1/1 and MAb 2/1 to peptide 306PKYVKQNTLKLATGMRNVPEKQT328 (●) and to the peptide epitopes contained within the polymers of poly T (□); poly T+B1 (⋄); poly T+B2 (◯) and poly T+B1+B2 (Δ) were determined by ELISA. The binding of normal mouse serum to the different peptide polymers was also determined (▪).

FIG. 10. Antigenic integrity of peptide polymers measured by T cell proliferative activity. The proliferative response of peptide monomer-primed lymph node T cells (A) or T cell clone 12V1 (B) against monomeric peptide T (□), poly T+B1 (⋄), poly T+B2, (●) or poly T+B1+B2 (Δ).

FIG. 11. Comparison of the immunogenic activity of monomeric and polymerised peptides. The titre of antisera raised in primary and secondary antibody responses against peptide monomers (open symbols) and peptide polymers (closed symbols) were determined in ELISA. Each symbol represents an individual animal and the mean antibody titre of each group is indicated by the solid line.

FIG. 12. Specificity of antisera raised to monomeric and polymeric peptides. The ability of antisera elicited by poly T+B1+B2 in a primary (Δ) and secondary (▴) antibody response to bind to analogue A and analogue B was measured in an ELISA assay. The binding of MAb 1/1 (◯), MAb 2/1 (●) and NMS (▪) to each of the analogues is also shown.

FIG. 13. Antibody isotype profiles elicited by polymeric peptides. The immunoglobulin isotypes obtained in the primary (open bars) and secondary (solid bars) antibody responses to poly T+B1, poly T+B2 and poly T+B1+B2 were examined by ELISA using isotype-specific reagents.

FIG. 14. Proliferation of clone T cell clone 4.51 in response to the helper epitope PKYVKQNTLKLA incorporating different cathepsin-sensitive sequences and co-polymerised with either serine, glutamic acid or acrylamide. Panel A: Proliferation induced by a polymer of the following acryloylated peptide sequences: VYLKY-PKYVKQNTLKLA co-polymerised with acryloylserine (●), GFLG-PKYVKQNTLKLA co-polymerised with acryloylserine (♦), PKYVKQNTLKLA co-polymerised with acrylamide ( [symbol] ),VYLKY-PKYVKQNTLKLA co-polymerised with acrylamide (▴),GFLG-PKYVKQNTLKLA co-polymerised with acrylamide (▾), monomeric peptide PKYVKQNTLKLATGMRNVPEKQT (□). Panel B: Proliferation induced by VYLKY-PKYVKQNTLKLA co-polymerised with acryloylglutamic acid (◯), GFLG-PKYVKQNTLKLA co-polymerised with acryloylglutamic acid (⋄), PKYVKQNTLKLA co-polymerised with acrylamide ( [symbol] ) monomeric peptide PKYVKQNTLKLATGMRNVPEKQT (□).

FIG. 15. Cytotoxic T-lymphocyte activity of polymer-primed cells against virus and peptide-pulsed targets. BALB/c mice were immunised subcutaneously with polymers (containing 10 μg of peptide) in CFA in the hind footpad or inoculated intranasally with infectious influenza virus A/Memphis/1/71 (1×10^{4,5} pfu). Lymph nodes of peptide-primed mice and spleen cells from virus infected mice were removed 7 and 21 days after inoculation and restimulated in vitro with either influenza virus-infected spleen cells or the CTL epitope, TYQRTRALV at a dose of 100μg/mL for 5 days. The CTL activity of cells obtained from animals primed with (▪) Virus, (♦) acryloyl TYQRTRALV co-polymerised with acryloyl serine, (●) acryloyl KKKTYQRTRALV co-polymerised with serine, (▴) acryloyl TYQRTRALV co-polymerised with acryloylglutamic acid, (▾) acryloyl KKKTYQRTRALV co-polymerised with acryloylglutamic acid, or (¤) acryloyl TYQRTRALV co-polymerised acrylamide was then measured in a 51Cr release assay using virus-infected targets (closed symbols) or noninfected targets (open symbols). Panel A: 51Cr released by CTL activity of cells restimulated in vitro with virus. Panel B: 51Cr released by CTL activity of cells restimulated in vitro with TYQRTRALV peptide.

FIG. 16. Antibody response induced by polymers of peptides at various doses and differing backbone compositions. BALB/c mice were inoculated either with acrylamide, or serine or glutamic acid-based co-polymers of the peptides GMRNVPEKQT and ALNNRFQIKGVELKS at doses of 5, 0.5 or 0.05 nmoles of peptide. Antisera were taken at day 30 after the primary inoculation and 12 days after the secondary inoculation and antibody titers determined by ELISA on plates coated with the peptide PKYVKQNTLKLATGMRNVPEKQT. Antibody titres are expressed as the titre obtained at an optical density of 0.25 units. Titers obtained following the primary (open symbols) and secondary (closed symbols) inoculations are indicated. The bar represents the mean titre for each group of antisera.

FIG. 17. Immunogenicity of the ST156/M52 heteropolymer. (A) Heteropolymer antisera to peptide ST156; (B) Heteropolymer antisera to peptide M52; (C) ST156 homopolymer antisera: recognition of peptide ST156; (D) M52 homopolymer antisera: recognition of peptide M52. Each line represent the result of one mouse. Initial immunisation Day 0, Primary boost Day 20, Secondary boost Day 30.

FIG. 18. Antibodies raised against the complete heteropolymer. Each line represents the result of one mouse.

### EXAMPLES

Section 1

Materials and Methods.

Unless otherwise stated chemicals were of peptide synthesis grade or its equivalent. O'Benzotriazole-N,N,N′,N′-tetra methyl -uronium-hexafluorophosphate (HBTU), 1-hydroxybenzotriazole (HOBt), diisopropylethylamine (DIPEA), N,N dimethylformamide (DMF), piperidine, trifluoroacetic acid (TFA), and 9-fluorenylmethoxycarbonyl (Fmoc) protected amino acids were obtained from Auspep Pty Ltd (Melbourne, Australia). Thioanisole, anisole, ethane dithiol (EDT), triisopropylsilane (TIPS) and acryloyl chloride were obtained from Aldrich (New South Wales, Australia). 1,8-diazabicyclo-[5.4.0] undec-7-ene (DBU) was obtained from Sigma Chemical Company (New South Wales, Australia) and acrylamide, ammonium persulphate, TEMED and tris (hydroxymethyl) amino methane (Tris) were obtained from BioRad (Richmond, Calif.). Phenol, dichloromethane (DCM) and diethylether were obtained from BDH (Poole, U.K.).

Solid phase Peptide Synthesis

Peptides were synthesised manually or using an automatic peptide synthesiser (either a Novasyn Crystal, Novabiochem, U.K. or a 9050 Plus PepSynthesiser (Milligen, Millford, Mass.). Standard solid phase peptide synthesis protocols for Fmoc chemistry were used throughout. Peptides were assembled as the C-terminal carboxyl or carboxyamide form using Novasyn KA 100 or KR 100 resins (Calbiochem-Novabiochem, New South Wales, Australia) respectively. Coupling was accomplished with HBTU/HOBt activation using 4 equivalents of amino acids and 6 equivalents of DIPEA. The Fmoc group was removed by 20% piperidine in Do or 2.5% DBU in DMF. Cleavage of peptides from the resin support was performed using reagent B (88% TFA, 5% phenol, 5% water, 2% TIPS) for 2 or 4 hours depending on the arginine content of the peptide. After cleavage the resin was removed by filtration and the filtrate concentrated to approximately 1 mL under a stream of nitrogen. After the peptide products were precipitated in cold ether, they were centrifuged and washed 3 times. The peptide precipitate was then dissolved in 5 to 10 mL of water containing 0.1% v/v TFA and insoluble residue removed by centrifugation.

N-acryloylation of peptides

Resins bearing peptides were swollen in a minimum amount of anhydrous, de-aerated DMF and acryloylated under nitrogen. After cooling on ice, a 20-fold molar excess of DIPEA in 0.5 ml DMF and a 10-fold molar excess of acryloyl chloride in 0.5 ml DMF were added to the resin. The mixture was stirred for 1 hour on ice and then for a further 1 hour at room temperature. The progress of acryloylation was monitored by the trinitrobenezene sulphonic acid (TNBSA) test When a negative TNBSA test was returned the resin was washed (5 ×in DMF, 3 ×in DCM and 3 ×in diethyl ether). The resin was than dried under vacuum.

Peptides P4 and P6 were assembled with Fmoc-Lys(Mtt)-OH (Calbiochem-Novabiochem, New South Wales, Australia) at their C-terminus and Boc-Pro-OH and Boc-Asp(OtBu)-OH at the N-terminus of P4 and P6 respectively. After assembly of the peptide, the Mtt group was removed with 1% TFA containing 5% TIPS in DCM and Fmoc-Ahx then coupled to the free ε-amino group using HBTU activation. The Fmoc group was removed from the Ahx with 2.5% DBU in DMF and the exposed amino group acryloylated as above.

Purification of Peptide Monomers

Purification of synthesised peptides was performed using a Pharmacia C18 Pep RPC column (1.6×10 cm) installed in a Fast Protein liquid Chromatography (FPLC) system (Amrad-Pharmacia Pty. Ltd., Victoria, Australia). Chromatograms were developed at a flow of 4 mL min⁻¹ using 0.1% TFA in water (solvent A ) and 0.1% TFA in acetonitrile (solvent B) as the limit buffer. All peptides were eluted with a linear gradient of 10-30% solvent B formed over 40 minutes. Analytical HPLC was carried out using a Vydac C18 column (4.6×300 mm) installed in a Waters HPLC system. Chromatograms were developed using solvent A and solvent B at a flow rate of 1 mL min⁻¹ and a 0-100% linear gradient of solvent B formed over 30 mins. Material eluted from columns was detected by determining the absorbance at 214nm or 280nm. ¹H NMR spectra of the N-acryloyl peptides were recorded at 300 MHz on a Bruker-AM300 spectrometer (in DMSO-d6). FAB mass spectra were recorded using a JOEL LMS-DX 300 spectrometer using an acceleration voltage of 3 keV with an FAB primary ion energy of 6 keV and emission current of 20 A. Xenon was employed as the bombardment gas. The scan range was 100 - 1500 m/z.

Co-Polymerisation of Acryloyl-Peptides with Acrylamide

General conditions for all polymerisations were as follows: a 1:50 molar ratio of acryloyl-peptide(s) and the co-monomer acrylamide (final concentration of 5% w/v) were mixed in degassed 6M guanidine-HCl (GuHCl) in 2mM EDTA and 0.5M Tris (pH 8.3). Polymerisation was initiated by addition of ammonium persulphate at a concentration of 2% of the molar concentration of acrylamide present and 5μl of a 20% v/v solution of TEMED. Polymerisation was allowed to proceed for 18 hours under nitrogen at room temperature.

Purification and Partial Characterisation of Peptide Polymers

Peptide polymers were isolated by gel permeation chromatography (GPC) using a column (1.6×60 cm) of Superdex 200 installed in a FPLC system. Chromatography was carried out at a flow rate of 3 mL min⁻¹ in 50mM NH₄HCO₃. All polymers eluted in the void volume. Peptide polymers isolated in this way were then lyophilised.

Amino acid composition of the peptide monomers and polymers was confirmed by amino acid analysis of purified material. Peptide material was hydrolyzed (0.001% w/v phenol in 6N HCI for 24 hours at 110° C.) and the hydrolysate derivatised with Fmoc-Cl. Amino acid analysis was carried out using a GBC Aminomate system using fluorometric detection.

Enzyme-linked immunosorbent assay (ELISA)

ELISAs were performed as described²³ using a solution (5 μg mL⁻¹) of peptide or peptide polymer to coat wells of flat-bottomed polyvinyl microtitre plates (Microtiter, Dynatech Laboratories, Va., U.S.A.). Bound antibody was then detected by incubation with horseradish peroxidase-conjugated (HRPO) rabbit immunoglobulin (Ig) directed against mouse Ig (DAKO, Denmark) or HRPO donkey Ig directed to sheep Ig (DAKO, Denmark) for 1.5h. After washing, substrate (0.2mM 2.2′-azino-bis 3-etthylbenztiazoline-sulphonic acid in 50mM citric acid pH 4.0 containing 0.004% v/v hydrogen peroxide) was added and after colour had developed the optical density (O.D.) at 405 nm was measured using a Titertek Multiskan MC (Flow Laboratories, Melbourne, Australia).

Inhibition ELISAs were carried out using a 1/1500 dilution of MAb 1/1 or anti-P8 antiserum incubated with known concentrations of inhibitor (soluble peptide or polymer) for 2 hours and then transferred to flat-bottomed polyvinyl microtiter plates coated with P5 or P8. Following overnight incubation, the plates were washed and the ELISA developed as above.

Antibodies

The preparation and properties of monoclonal antibody MAb 1/1 has been described elsewhere¹⁹. MAb 1/1 was raised against the synthetic peptide representing the C-terminal 24 residues (³⁰⁵CPKYVKQNTLKLATGMRNVPEKQT³²⁸) of the heavy chain (HA₁) of the hemagglutinin of influenza virus A/Memphis/1/71 (H3) and is specific for the B cell determinant RNVPEKQT ²⁰. Hyperimmune serum (HIS) was raised in sheep to luteinising hormone releasing hormone (LH—RH; P8).

Synthesis of acryloyl peptides

Peptide sequences varying in length from 4 to 23 residues (Table 1) were assembled by solid phase peptide synthesis employing Fmoc chemistry. In general, 6-amino hexanoic acid (Ahx) was introduced as the last “residue” and the N-terminus then acylated by acryloylation. In this way Ahx functions as a spacer to distance the acryloyl group from the peptide.

TABLE 1
Amino acid sequences of peptides used

| | Desig- | |
|---|---|---|
| Amino Acid Sequences^{a} | nation | Origin |
| TLKLATG^{b} | P1 | Influenza virus |
| GMRNVPERKQT | P2 | Influenza virus |
| ALNNRFQIKGVELKS | P3 | Influenza virus |
| PKYVKQNTLKLATG | P4 | Influenza virus |
| PKYVKQNTLKLATGMRNVPEKQT | P5 | Influenza virus |
| DCTLIDDALLGDPH | P6 | Influenza virus |
| TYQRTRALV | P7 | Influenza virus |
| SQHWSYGLRPG | P8 | LHRH^{c} |
| DRAAGQPAGDRAAGQPAGDR | P9 | Malaria parasite |
| (NANP)₅ | P10 | Malaria parasite |
| FNNTVSFWLRVPKVSASHLE | P11 | Tetanus toxin |
| QYIKANSFIGITEL | P12 | Tetanus toxin |
| LRRDLDASREAKKQVEKALE | P13 | Group A |
| | | Streptococcus |
| YIYADGKMVNEALVRQGLAK | P14 | Staphylococcal |
| | | nuclease |
| DLIAYLKQATAK | P15 | Pigeon cytochrome C |
| GFGA | P16 | model peptide |

^{a}single letter code
^{b}all peptides were assembled as the carboxyamide except P2, P5 and P7 which were synthesised with a free —COOH at the C-terminus
^{c}luteinising hormone releasing hormone

Three techniques for acryloylating resin-bound peptides were evaluated. Two methods involving the coupling of acrylic acid to the N-terminus by either a symmetrical anhydride route¹⁷ or by using 0-benzotriazole-N,N,N′,N′-tetramethyl-uronium-hexafluorophosphate and N-hydroxybenzotriazole activation gave poor yields of pure N-acryloyl peptides. Consistent and high yields of pure N-acryloyl peptides are, however, obtained by acylation of the peptide N-terminal amino group with acryloyl chloride (Scheme 1).

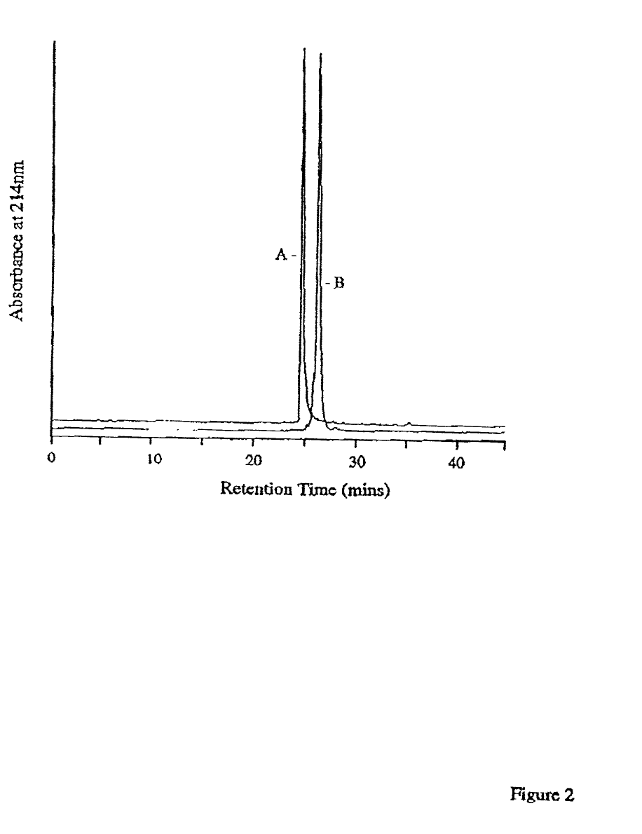

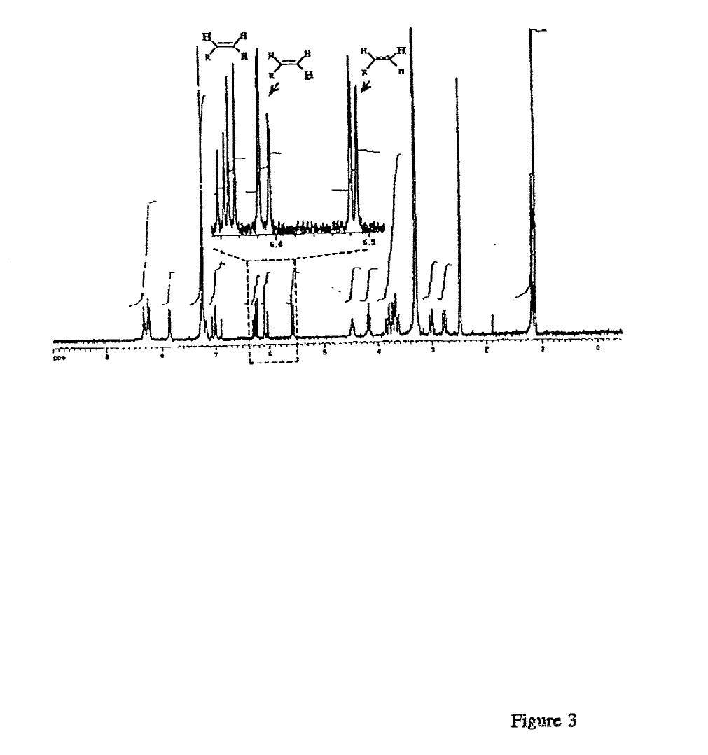

As can be seen from FIG. 2 the acryloylation of peptide yields material with an increased retention time compared to the non-acryloylated peptide when measured by reverse phase chromatography. Reagent B³⁶ was used for the cleavage of N-acryloyl peptides from the resin support and side chain deprotection because the use of reagent R (peptide NH₂-Gly-Phe-GlY-Ala, when acryloylated with acryloyl chloride and then cleaved with reagent R (TFA : thioanisole : anisole : ethane dithiol 90:5:3:2) gave an m/z value 124 Daltons greater than expected by FAB mass spectrometry, a result consistent with thioanisole addition to the double bond) resulted in the addition of thioanisole to the double bond. The crude acryloylated peptides were purified by reverse phase chromatography and gave the expected amino acid analysis. Typical ¹H NMR resonances for the acryloyl group were obtained with a variety of N-acryloylated peptides. A typical spectrum is shown (FIG. 3) for the model peptide N-acryloyl GFGA (P16).

Although the majority of peptides are N-terminally acryloylated, peptides P4 and P6 have been synthesised with the acryloyl group at their C-terminus. This was achieved by the acryloylation of the ε-amino group of lysine which was introduced at the C-terminal position as Fmoc-Lys-(Mtt)-COOH The ability to synthesise N- or C-terminally acryloylated peptides allows for opposite orientations of a peptide within a polymer.

Polymerisation of synthetic peptides

A simple protocol (Scheme 1), similar to that used in routine preparation of polyacrylamide gels, was adapted to assemble linear polyacrylamides bearing pendant peptides. This procedure yields high molecular weight, water soluble peptide polymers. The polymerisation of short (tetra or penta) peptides has been reported^{17, 34} using similar methodology but in those studies high temperatures, which may be detrimental to biological activity, were used and the side chain protecting groups, which can introduce potential solubility problems, were left in place during the polymerisation process. The assembly of polyacrylamides bearing sialic acid groups has also been reported³⁵.

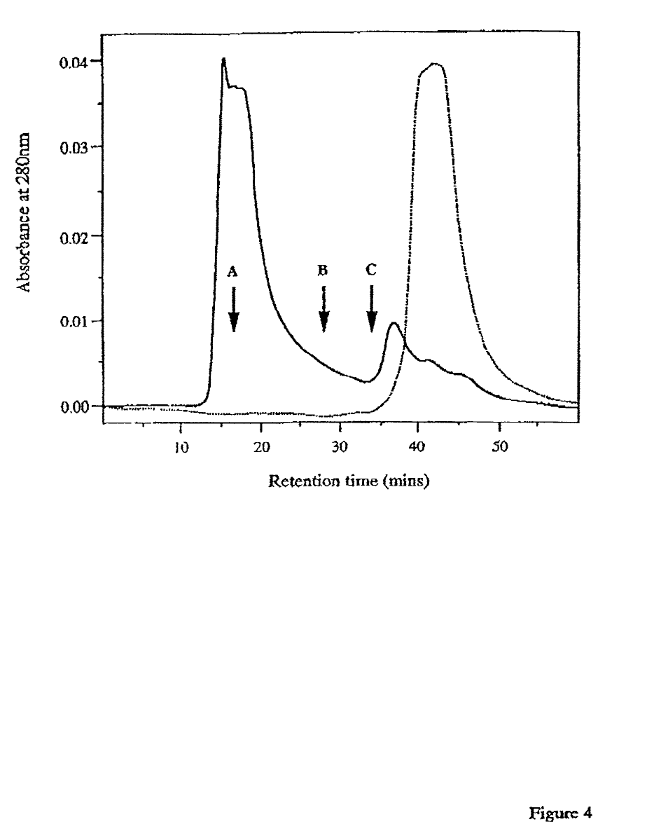

The polymerisation reaction was routinely carried out in degassed 6M guanidine-HCl (GuHCI) and 2mM EDTA in 0.5M Tris (pH 8.3) although solvents such as 1.5M Tris-HCl (pH 8.8) and 8M urea were also successfully employed. A 50-fold molar excess of acrylamide over the amount of acryloylated peptide was introduced to allow stretches of polyacrylamide to be interspersed between the various acryloyl peptide units with the aim of minimising steric interactions between the peptide chains and to maximise water solubility of the overall polymer. This was not always necessary because peptides P2, P9, P10 and P13 incorporating Ahx were polymerised successfully in the absence of added acrylamide. Polymerisation was initiated by addition of ammonium persulphate and N,N,N′,N′-tetramethylethylenediamine (TEMED) and the reaction mixture allowed to stand for 18 hrs at room temperature under N₂. Time course analysis of the polymerisation process indicated that incorporation of peptide into polymer is rapid with evidence that some peptide is incorporated into polymer within 15 minutes. FIG. 4 shows high molecular weight peptide-containing material formed when CH₂═CHCO—P8 but not when NH₂—P8 was subjected to the polymerisation conditions. Each of the peptide polymers examined eluted in the void volume of a column (1×30 cm) of Superose 6 which has an exclusion molecular weight of approximately 4×10⁷ Da for globular structures. Amino acid analysis of the polymers indicated that 80 to 97% of acryloylated monomeric peptides were incorporated into the polymer. The data also indicate that different peptides may be incorporated into the same polymeric structure and the ratios of these, when co-polymerised in equimolar amounts, is approximately equivalent (Table 2). This technique was successfully applied to the co-polymerisation of the following combinations of peptides: P1+P3; P2+P3; P1+P2+P3; P1+P4; P2+P4; P1+P2+P4; P8+P11; P9+P11; P9+P10; P9+P10+P11; P1+P3+P14+P15; P1+P14 and P1+P15.

Antigenicity of synthetic peptide polymers

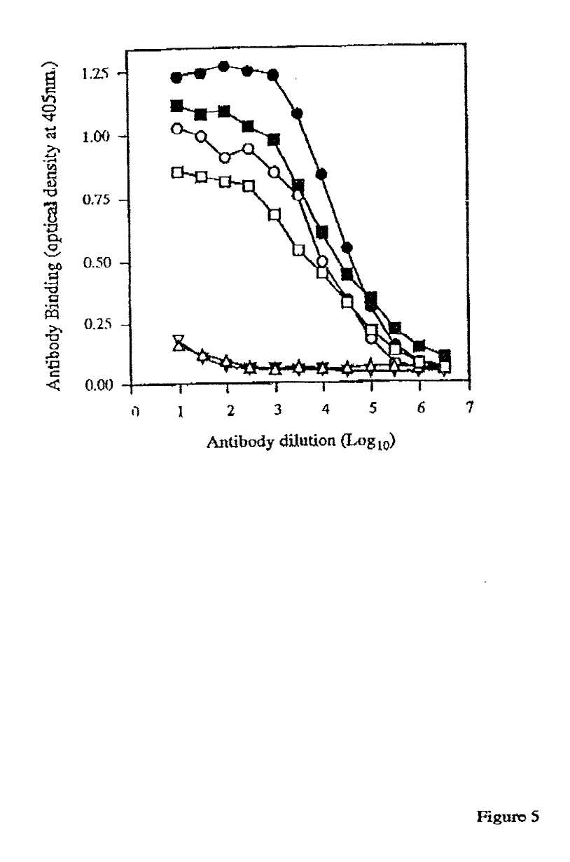

A potential concern of free radical-induced polymerisation of unprotected peptide monomers is that their side-chain functions may be altered affecting their antigenicity and immunogenicity. To examine this possibility, polymerised P8 and polymerised P2, both of which contain side-chain functional groups likely to be affected by free radicals, were coated on plastic microtitre trays and probed with antiserum directed against P8, or a monoclonal antibody (MAb 1/1) which is specific for P2^{19,20}. The results (FIG. 5) demonstrate that each antibody was capable of binding efficiently to determinants within the polymers indicating that the epitopes remain antigenically intact despite exposure to free radical.

An advantage of polymerising synthetic peptides is that the multiple copies of a peptide that are present in a single molecule could confer

TABLE 2
Amino acid composition of peptide polymers

| | Polymers of: | | | | | | | |
|---|---|---|---|---|---|---|---|---|
| | P2 | | P8 | | P6 + P7 | | P1 + P3 + P14 + P15 | |
| Amino | Residues | Residues | Residues | Residues | Residues | Residues | Residues | Residues |
| acid^{a} | found | expected | found | expected | found | expected^{b} | found | expected^{b} |
| D or N | 0.9 | 1 | 0 | 0 | 2.8 | 3 | 5.2 | 5 |
| E or Q | 2 | 2 | 1.1 | 1 | 1 | 1 | 5.6 | 5 |
| S | 0 | 0 | 1.9 | 2 | 2 | 2 | 1.3 | 1 |
| H | 0 | 0 | 0.9 | 1 | 2.2 | 2 | 0 | 0 |
| G | 0.9 | 1 | 1.8 | 2 | 3.2 | 3 | 4.2 | 4 |
| T | 1.1 | 1 | 0 | 0 | 0.9 | 1 | 3.3 | 3 |
| A | 0 | 0 | 0 | 0 | 0.9 | 1 | 8.3 | 8 |
| P | 1 | 1 | 1.0 | 1 | 1.9 | 2 | 0 | 0 |
| Y | 0 | 0 | 0.9 | 1 | 1.3 | 1 | 3.3 | 3 |
| R | 1.1 | 1 | 1.0 | 1 | 1.0 | 1 | 2.3 | 2 |
| V | 1 | 1 | 0 | 0 | 0 | 0 | 0 | 0 |
| M | 0.9 | 1 | 0 | 0 | 0 | 0 | 0.8 | 1 |
| I | 0 | 0 | 0 | 0 | 0.9 | 1 | 3.2 | 3 |
| L | 0 | 0 | 1 | 1 | 3.9 | 4 | 8.4 | 8 |
| F | 0 | 0 | 0 | 0 | 0 | 0 | 1.5 | 1 |
| C | 0 | 0 | 0 | 0 | 0.9 | 1 | 0 | 0 |
| K | 1 | 1 | 0 | 0 | 0 | 0 | 7.4 | 7 |

^{a}Single letter code
^{b}assuming stoichiometric incorporation of individual peptides into polymer

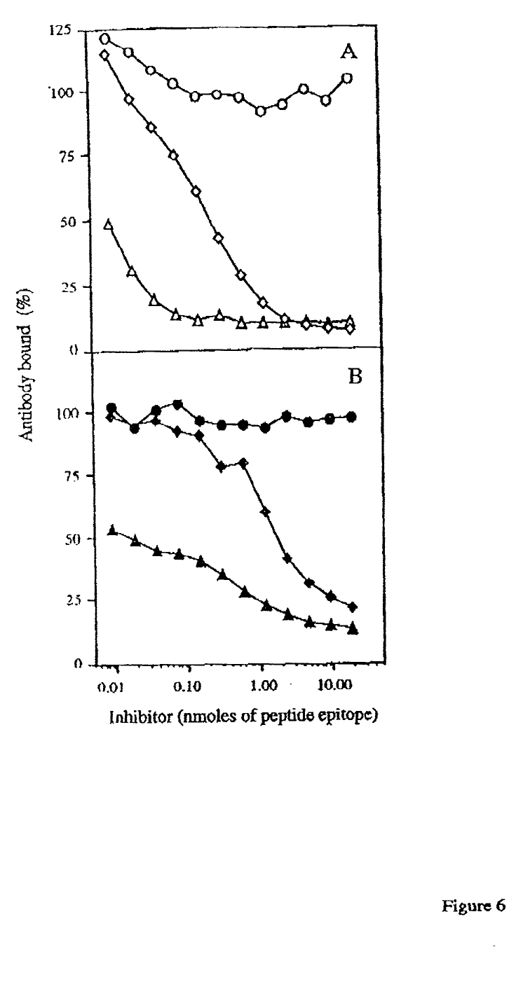

enhanced antigenicity over peptide monomers ₃₇. To evaluate this, an inhibition ELISA, which is independent of any difference in the ability of the antigens to bind to the microtitre plate, was used to compare the antigenic properties of monomeric and polymeric peptides. P2 and polymerised P2 were used to inhibit the binding of MAb 1/1 to P5-coated microtitre plates and similarly, P8 monomer and polymerised P8 were used to inhibit the binding of the anti-P8 antiserum to P8-coated plates. As can be seen in FIG. 6, polymeric peptides are more antigenic than peptide monomers. A polymer of P4 (which does not contain epitopes recognized by MAb 1/1 or anti-P8 antiserum) did not inhibit the binding of these antibodies to their relevant peptides indicating that increased inhibition caused by the peptide polymers is a consequence of the presentation of multiple copies of each peptide determinant. The results in FIGS. 5 and 6 demonstrate not only that polymerised peptides retain antigenic integrity but are more antigenic than the corresponding peptide monomer, presumably because multiple copies of the same antigenic determinant allows high avidity interaction with antibody.

Conclusion

The synthesis and polymerisation of unprotected acryloyl peptides provides a practical generic method for the synthesis of peptide macromolecules. High yields of acryloyl peptides are obtained by using acryloyl chloride to derivatise the N-terminus of protected peptides still attached to the resin support followed by cleavage and deprotection using reagent B. The inclusion of 6-amino hexanoic acid as a spacer between the peptide and the acryloyl group allows the polymerisation of certain peptides in the absence of acrylamide. Free radical polymerisation does not affect the antigenic integrity of peptides and peptide polymers are more antigenic than monomeric peptides. An advantage of this approach is that any number of the same or different acryloyl peptides can be assembled into a polymer with the expectation that the overall antigenic activity of the construct will be largely determined by the nature of the peptide units which are pendant from a hydrocarbon chain. By polymerising a mixture of B-cell and T-cell peptide epitopes it will be possible to assemble a construct in which all or most of the important epitopes of a pathogen or of several pathogens are represented. This is particularly important for those organisms such as the malaria parasite, HIV and influenza virus and group A streptococci which occur as serologically diverse strains.

Section 2

Materials and Methods

Chemicals.

Unless otherwise stated chemicals were of analytical grade or its equivalent. N,N′-dimethylformamide (DMF), piperidine, trifluoracetic acid (TFA), O'Benzotriazole-N,N,N′,N′-tetra methyl-uronium-hexafluorophosphate (HBTU), 1-hydroxybenzotriazole (HOBt) and diisopropylethylamine (DIPEA) were obtained from Auspep Pty Ltd (Melbourne Australia). Phenol, triisopropylsilane and acryloyl chloride were from Aldrich (Milwaukee, Wis.) and trinitrobenzylsulphonic acid (TNBSA) from Fluka (Switzerland); 1,8-diazabicyclo-[15.4.0]undec-7-ene (DBU) was obtained from Sigma and acrylamide, ammonium persulphate and N,N,N′,N′-tetramethylethylenediamine (TEMED) were obtained from BioRad (Bio-Rad Laboratories Pty., Ltd., Australia) and dichloromethane (DCM) and diethylether were from BDH (Poole, U.K.).

Peptide Synthesis

Peptides were either synthesised manually or using an automatic peptide synthesiser (either a Novasyn Crystal, Novabiochem U.K. or a 9050 Plus, Milligen). Fmoc chemistry was used throughout and peptides were assembled as the C-terminal carboxyl or carboxamide form. Fmoc amino acids were either incorporated into the peptide chain as the activated O-pentaflurophenyl ester or by using the free acid with HBTU and HOBT present in equimolar amounts and DIPEA present at a 1.5-fold molar excess. Piperidine (20% in DMF) or DBU (2.5% in DMF) was used to remove the Fmoc group following incorporation of the amino acid residue. Fmoc-6-amino hexanoic acid (Ahx) was manually coupled to the N-terminus of peptides using HBTU, HOBT and DIPEA. The Ahx group acted to space the peptide from the polymer backbone. All coupling reactions carried out manually were monitored using the TNBSA test¹⁸.

Acryloylation of the N-terminus of resin-bound protected peptides was carried out manually in anhydrous, de-aerated DMF at 0° C. under nitrogen. A 20-fold molar excess of DIPEA (in 0.5 ml DMF) and a 10-fold molar excess of acryloyl chloride (in 0.5 ml DMF) were added and the reactants mixed for 1 hour on ice, then for a further hour at room temperature. The progress of acryloylation was monitored by the TNBSA test. At completion of peptide assembly, the supports with peptide attached were washed sequentially in DMF, DCM and diethyl ether and then dried under vacuum.

Peptides were cleaved from the resin support and the side chain protecting groups removed using reagent B (TFA, phenol, water and triisopropylsilane in the ratio 88:5:5:2) at room temperature under nitrogen for either 2 or 4 hours, depending on the arginine content of the peptide. After partial evaporation of the peptide-containing filtrate under a stream of nitrogen, the crude peptide was precipitated in, and then washed three times with, cold ether.

Peptide Purification

Peptides were purified by reversed phase chromatography on a PepRPC column (1.6 ×10cm) installed in an FPLC system (Amrad/Pharmacia Pty. Ltd) and the chromatogram developed at a flow rate of 4ml/min using 0.1% TFA in H₂O and 0.1% TFA in CH3CN as the limit solvent. Analytical HPLC was carried out using a Vydac C4 column (4.6×300 mm) installed in a Waters HPLC system and developed at a flow rate of 1ml/min using similar solvents.

Authenticity of the purified peptides was monitored by Analytical RP-HPLC, amino acid analysis and the presence of the -ene group monitored by ¹H NMR; spectra were obtained at 300MHz on a Bruker AC 300 spectrometer.

Polymerisation of N-acryloylated Peptides

Polymerisation of N-acryloylated peptides was carried out under nitrogen in degassed 0.5M Tris (pH 8.3) containing 6M guanidine-HCl and 2mM EDTA. Assembly of polymers of peptides was typically carried out with 3,μmoles of N-acryloyl-peptide(s) and a 50-fold molar excess of acrylamide. Polymerisation was initiated by addition of ammonium persulphate (2% of the total number of moles of acrylamide) and TEMED (5μl of a 1.32μM solution) and the reaction mixture allowed to stand for 18 hrs at room temperature.

Peptide polymers were isolated using a column (1.6×60cm) of HiLoad Superdex 200 prep grade (Amrad/Pharmacia Pty. Ltd.) installed in an FPLC system and the chromatogram developed at a flow rate of 3ml/min using 50mM ammonium bicarbonate. Molecular weight estimations were carried out using a Waters HPLC system with a Superose 6 HR column (1×30 cm) and developed at a flow rate of 0.5 ml/min in 50mM ammonium bicarbonate.

Monoclonal Antibodies

The preparation and properties of monoclonal antibodies (MAbs) 1/1and 2/1 have been described elsewhere¹⁹. These MAbs were raised against the synthetic peptide ³⁰⁵CPKYVKQNTLKLATGMRNVPEKQE³²⁸ representing the C-terminal 24 residues of the heavy chain (HA₁) of the hemagglutinin of influenza virus A/Memphis/1/71. MAb 1/1 is specific for the B cell determinant RNVPEKQT (B₁) and requires ³²²N, ³²⁵E and ³²⁷Q for binding, whereas MAb 2/1 recognises the B cell determinant LKLAT (B₂) and has an absolute requirement for each of these five residues²⁰.

T Cells

T cell clone 12V1was raised in mice immunized with purified hemagglutinin light chain (HA₂) and proliferates in response to the peptide ALNNRFQIKGVELKS derived from HA₂. The derivation, properties and maintenance of this clone have been described elsewhere²¹. Prior to use, the cultured cells were passaged over Isopaque-Ficoll gradients^{22,23}.

Lymph node T cells were derived from pooled inguinal and popliteal lymph node cell suspensions of mice primed seven days previously with peptide. T cells were enriched by passage through nylon wool columns²⁴.

Immunization Protocols

Female Balb/c mice, 6 to 8 weeks of age, were used. For antibody studies, animals were inoculated i.p. with synthetic peptide in monomeric or polymeric form and emulsified in complete Freund's adjuvant (CFA). Mice received 5 or 0.5 nmoles of monomeric peptide or the equivalent number of moles of determinant within a polymer. For T-cell studies, mice were inoculated s.c. in the hind footpads with a total of 10 nmoles of T-cell determinant in CFA.

Antibody Binding Assays

Enzyme-linked immunosorbent assays (ELISAs) were performed as described²³ using microtitre plates coated with a solution of 5μg/ml peptide or polymer antigen. Bound antibody was detected by incubation with horseradish peroxidase-conjugated rabbit immunoglobulin (Ig) directed against mouse Ig (DAKO, Denmark) for 1.5h followed, after washing, with substrate (0.2mM 2,2′-azino-bis 3-ethylbenzthiazoline-sulphonic acid in 50mM citric acid pH4.0 containing 0.004% v/v hydrogen peroxide). The absorbance at a wavelength of 405nm was determined using a Labsystems Multiscan Multisoft microplate reader (Pathtech Diagnostics Pty. Ltd., Aust.) and antibody titres were expressed as the reciprocal of the antibody dilution giving an absorbance of 0.25, which corresponds to at least five times the background value.

Isotyping of Mouse Sera

Sera taken from mice that had received two doses of a particular antigen were pooled and the isotype profile of the antibodies examined by ELISA. Dilutions of antisera were added to peptide ³⁰⁶PKYVKQNTLKLATGMRNVPEKQT³²⁸-coated plates and incubated overnight. After washing the wells, rabbit antisera to mouse IgM, IgG1, IgG2a, IgG2b, IgG3 or IgA (ICN Pharmaceuticals Inc., Costa Mesa, Calif.) was added and allowed to bind for 4h. Plates were again washed and a 1/400 dilution of horseradish peroxidase-conjugated swine anti-(rabbit Ig) was added. After 1.5h, the trays were again washed, substrate added and the absorbance determined as above.

T Cell Proliferation Assays

T cells were cultured (at a concentration of 3×105 cells/well for lymph node T cells and 1×10⁴/well for the T-cell clone) in 96-well nmicrotitre trays (Nunc, Denmark) in the presence of syngenic γ-irradiated (2,200 R, ⁶⁰Co source) spleen cells as a source of antigen presenting cells, together with antigen in a total volume of 250μl^{22, 23}, The culture medium was RPMI 1640 supplemented with 10% heat inactivated (56° C., 30min) fetal calf serum, 2mM glutamine, 2mM sodium pyruvate, 0.1mM 2-mercaptoethanol, 30μg/ml gentamicin, 100 I.U./ml penicillin and 100μg/ml streptomycin. Cultures were incubated for 4 days at 37° C. in an atmosphere of 5% CO₂ with 1μCi/well ³H-thymidine present during the final 18hr. Cells were then harvested onto glass fibre filters and incorporation of ³H-thymidine was measured on a Hewlett Packard Matrix 9600 direct β-counter.

Results

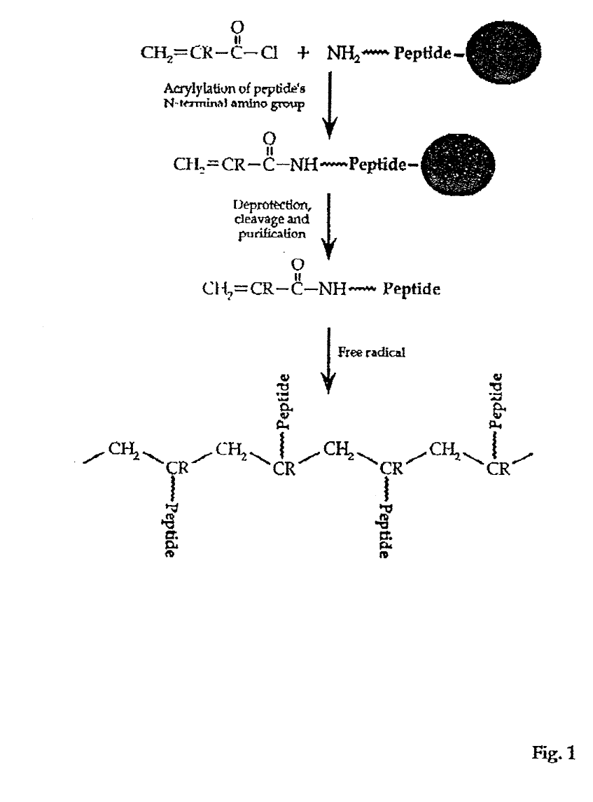

Polymerisation of N-acryloyl-peptides. In this study a simple method for polymerising individual T- and B-cell determinants into a single construct was developed to enable strong T-cell determinants to be efficiently co-delivered with B-cell determinants in a multimeric immunogen. To evaluate the potential of is technology we have used the well characterised²⁵ determinants for B cells and helper T cells which reside within peptide 306-328 which represents the 24-residue C-terminal sequence of the heavy chain (HA₁) of influenza virus A/Memphis/1/71 (H₃ subtype) hemagglutinin. This peptide contains two B-cell determinants encompassed by the sequences GMRNVPEKQT (B₁) and TLKLATG (B₂) and a helper T-cell determinant KYVKQNTLKL that overlaps with B₂. A second T cell determinant ALNNRNFQIKGVELKS (T), located elsewhere on the viral hemagglutinin within the light chain (HA₂) has been reported to be a more potent inducer of B cell help for the production of antibody directed to B₂than is the native overlapping helper determinant²⁶. Polymers of T alone or in combination with B₁, B₂ or B₁together with B₂ (B₁+B₂) were prepared using the scheme shown in FIG. 1. Peptides were synthesised with an Ahx group at the N-terminus followed by acylation with the acryloyl group. Polymerisation of N-acryloylated peptides was then carried out in guanidine-HCI to aid solubility. A 50-fold molar excess of acrylamide was also added to act as a spacer along the polymer backbone. The reaction is comparable to the formation of polyacrylamide gels in the absence of crosslinking reagent. Amino acid analysis of the peptide polymers made from equimolar amounts of different peptides confirmed the presence of the relevant peptides in approximately equimolar ratios: in poly (T +B₁) the ratio of T to B₁ was 1:1.3; in poly (T +B₂) the ratio was 1:1.2; in poly (T +B₁ +B₂) the ratio was 1:1.4:1.1.

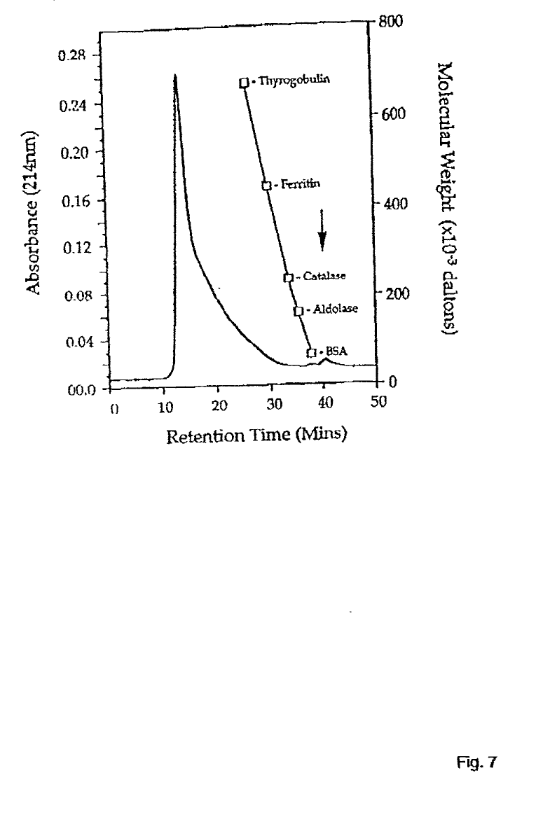

Some of the chromatographic properties of the polymers produced using this protocol were investigated by gel permeation chromatography and FIG. 7 shows the elution profile of poly (T +B₁ +B₂) from a column (1×30 cm) of Superose 6 HR. This profile was representative of that obtained with each of the different polymeric immunogens. The polymer eluted in the void volume of the column, a position quite distinct from that of monomeric peptides. In all cases, very little non-polymerised peptide remained after the polymerisation process.

Antigenicity of Peptides Exposed to Free Radicals

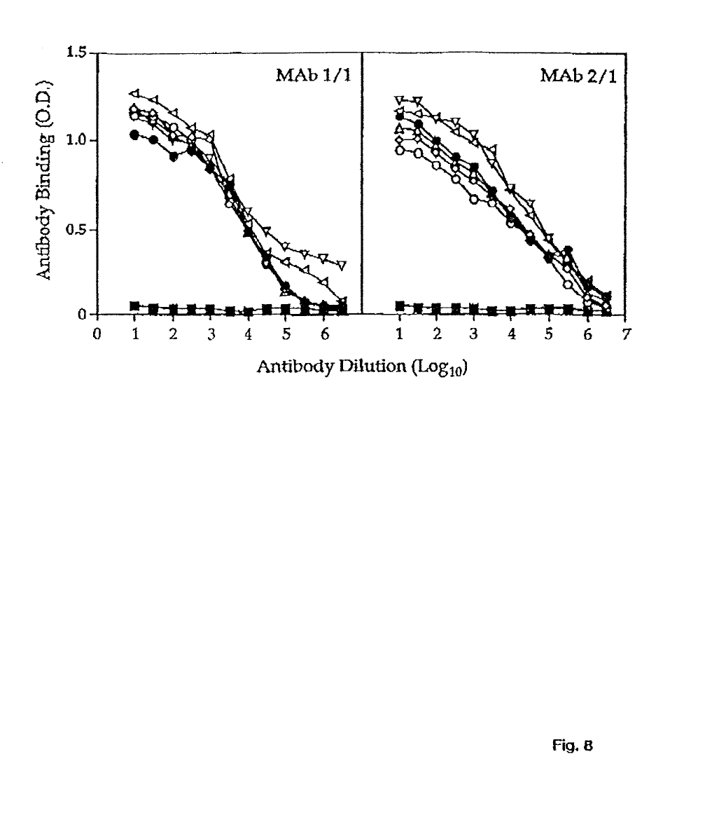

The polymerisation procedure utilises free radicals which are potentially capable of damaging functional groups of proteins. To determine whether the conditions chosen for the free radical-induced polymerisation of synthetic peptides described here had any detrimental effect on the antigenic activity of the peptide epitopes, monomers of peptide 306-328, lacking the acryloyl group were exposed to various concentrations of ammonium persulphate in the presence of acrylamide. The peptide was then separated from the acrylamide polymer and used to coat wells for an ELISA assay. The ability of these treated peptides to bind MAbs 1/1 and 2/1, which have specificity for B₁ and B₂ respectively, is shown in FIG. 8. These results indicate that at each of the concentrations of persulphate tested (persulphate to peptide ratios varied between 0.1:1 and 1,000:1), there is little or no effect on antigenic activity. The ratio of persulphate to peptide utilised in the polymerisation procedure is well below this range at 0.02:1.

Antigenic Integrity of Synthetic Polymeric Immunogens

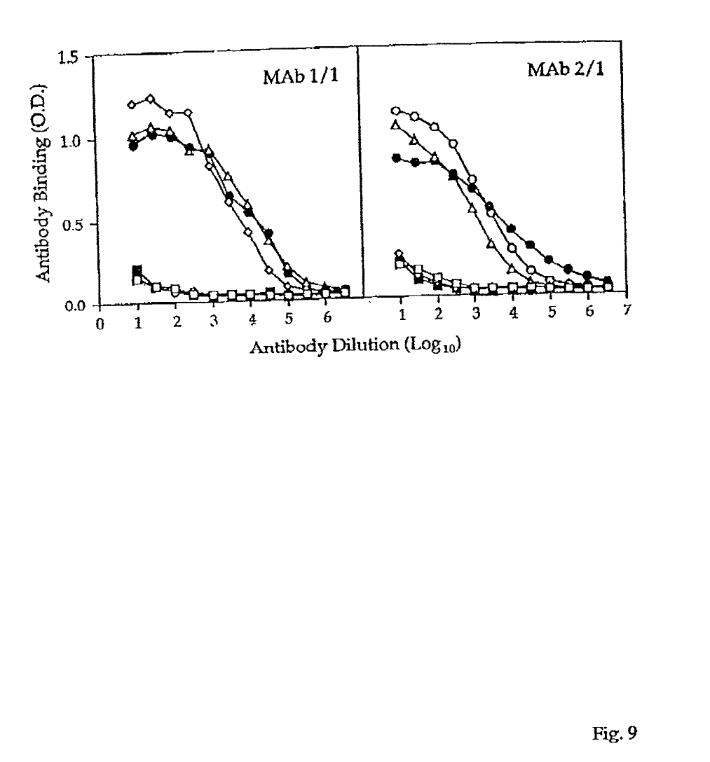

To determine whether the B-cell determinants within the polymeric immunogens were accessible to antibody raised against the monomeric peptide 306-328, polymers were coated onto wells of a microtitre tray and probed with MAb 1/1 or 211 by ELISA (FIG. 9). MAb 1/1 was capable of binding efficiently to polymers containing the B₁ determinant displaying similar binding characteristics with these as with the monomeric peptide. MAb 2/1 also bound well to polymers containing the B₂ determinant.

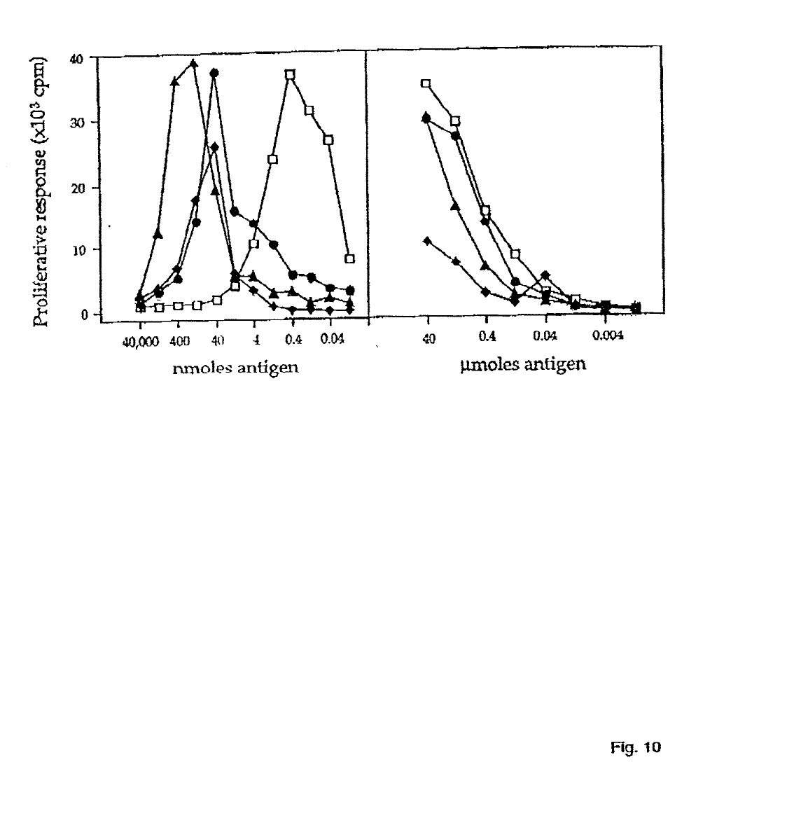

The integrity of the T-cell determinant within the polymeric immunogens was also examined. FIG. 10 shows the ability of different polymers to stimulate the in vitro proliferation of T cell clone 12V1 (FIG. 10A) which is specific for the T sequence ALNNRFQIKGVELKS²¹. High dose antigen suppression, typically observed in this system^{26, 27}, was evident with each antigen and comparison of the dose required for peak proliferative responses demonstrated that the polymers had greatly reduced capacity to stimulate the clone relative to the monomeric T cell determinant peptide. This may indicate that the determinant is not readily processed from the polymers for presentation to the clone. Such large differences in efficiency of recognition were not, however, observed with a polyclonal population of lymph node T cells raised against the monomeric T-cell determinant peptide (FIG. 10B). In this instance, the T cells recognised the T monomer and poly (T+B₂) with equal efficiency, and poly (T+B₁+B₂) to a slightly less extent. Poly (T+B₁) was the least well recognised.

Immunogenicity of the Polymeric Immunogens

The relative immunogenic properties of monomeric and polymeric immunogens were assessed by comparing the antibody levels elicited by monomeric peptide 306-328 and a polymer of the peptide 306-319 which contains the B_{z} determinant and the overlapping T cell determinant, KYVKQNTLKL^{27, 28}. These two immunogens were tested in mice at a dose of 5nmoles of peptide epitope and sera were taken during the primary and secondary responses. At this dose the monomeric peptide gave a very weak primary antibody response which rose some 10-fold following the second inoculation. In contrast, the polymeric immunogen gave a substantial primary response (approximately 30-fold higher mean titre than the monomer) and this was increased a further 10-fold after a second dose to give an overall 30-fold enhancement of antibody production.

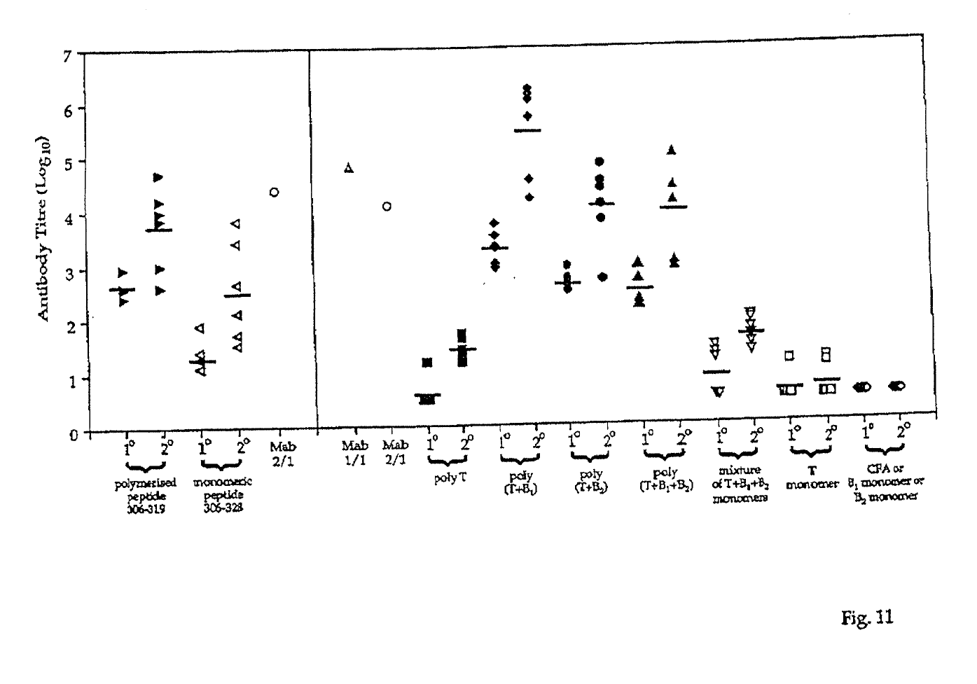

The multi-component polymers, based on the T sequence together with B₁ or B₂ or both, were also inoculated into mice and the antibody response determined. Doses of 0.5nmoles and 5nmoles elicited similar levels of antibody and the data obtained for the 0.5nmole dose only is reported (FIG. 11B). Not unexpectedly, the individual monomeric B₁ or B₂ determinants yielded no antibody when inoculated into mice, but the same determinants when co-polymerised with the T sequence were potent immunogens. The levels of antibodies elicited in response to a single dose of polymer (panel B) were comparable or higher than those achieved after two doses of peptide 306-328 monomer (panel A) and those obtained after two doses of the polymers were 50 to 100-fold greater than with the peptide 306-328. Of the three polymeric immunogens containing the T sequence, the poly (T+B₁) construct elicited the highest levels of antibody, higher on average than the titres of MAbs in the control ascitic fluid preparations.

Specificity of Antibody to the Polymeric Immunogen containing two B-cell Determinants

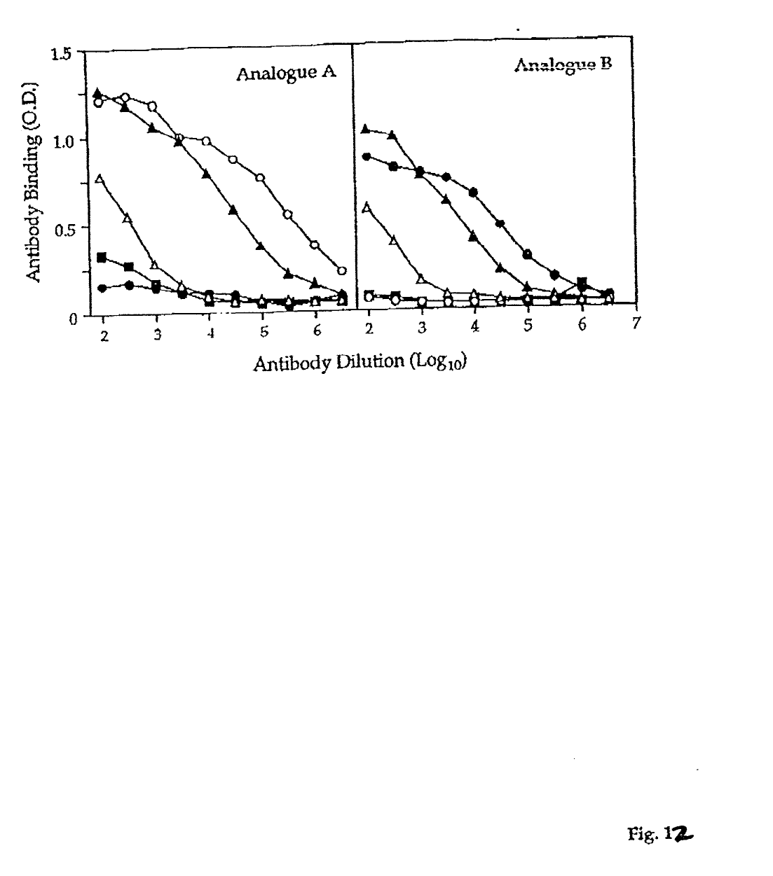

In FIG. 11 the levels of antibody obtained with the poly (T+B₁+B₂) immunogen did not amount to the sum of the antibodies obtained with poly (T+B₁) plus poly (T+B₂). This is not unexpected if the ratio of T-cell determinant to B-cell determinant influences the overall amount of antibody produced. However, as the poly (T+B₁+B₂) immunogen elicited very similar titres to the poly (T+B₂) immunogen, it raised the question as to whether both B-cell determinants were recognised within poly (T+B₁+B₂) or whether one became immunodominant at the expense of the second. To test this, the antisera to poly (T+B₁ +B_{z}) were tested in ELISA against peptides referred to as analogue A and analogue B, which have a similar sequence to peptide 306-328 but contain only one of the two B-cell determinants of this peptide, B₁and B₂ respectively,¹⁹. FIG. 12 shows that sera taken during the primary and secondary response to poly (T+B₁+B₂) were capable of binding to both of these analogue peptides indicating that antibodies specific for both B₁and B₂ were elicited by this multicomponent polymer.

Isotype of antibody elicited by the polymeric immunogens

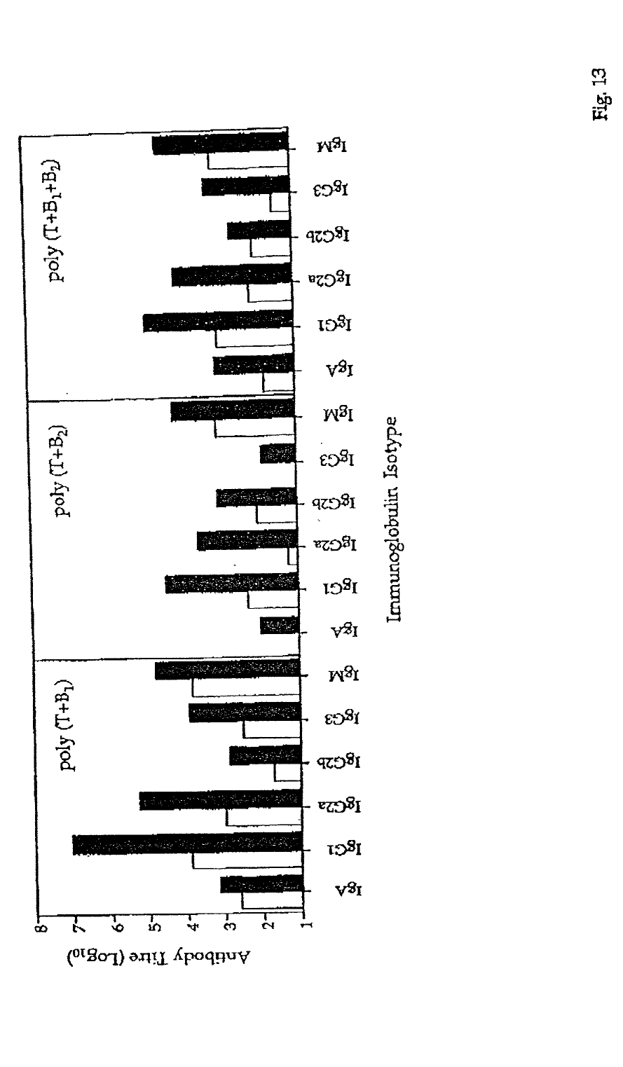

Certain polyvalent immunogens can trigger B cells in a T-independent manner and this interaction results in antibody of a restricted isotype profile with the dominant subclass being IgM. The isotype profiles of serum antibody produced in response to poly (T+B₁), poly (T+B₂) or poly (T+B₁+B₂) were therefore examined to determine whether these polymers were restricted in the isotypes of antibody that they could elicit. FIG. 13 shows that a range of different antibody isotypes are produced in response to each of the three polymers.

Discussion

Defined antigens rather than crude antigen preparations will form the third generation of vaccines and the difficulties associated with eliciting an immune response in all individuals against multiple serotypes of particular pathogens will require the application of focussed rather than empirical approaches. Molecular immunological techniques have allowed the identification of many epitopes that are involved in immune responses against disease-causing organisms. Foremost amongst the technologies which have been used to identify these epitopes is peptide synthesis and we are now at the point where methods of delivering these epitopes to the immune system, in a way which will elicit a useful immune response, need to be developed. Here we have approached this problem by developing a method for the assembly of polyvalent homo- and heteropolymers of B- and T-cell epitopes.

The method we describe results in very large polymeric species which elute in the void volume of a column whose exclusion molecular weight for globular structures is approximately 4×10⁷ Daltons. It is not clear whether such large structures are required for enhanced immunogenicity but in theory uptake of such polymers by antigen presenting cells would allow presentation of the determinants in high copy number per cell. The form of these “synthetic proteins” may also confer greater serum stability on the determinants compared to free monomeric peptides. By introducing chain transfer reagents into the polymerisation protocol, it is possible to control the size of the polymer thereby allowing an examination of the effect of size on immunogenicity.

Multicomponent polymers of a T-cell determinant sequence together with the B₁and/or B₂ determinants were successfully constructed and shown to be antigenically intact. Results from subsequent experiments in which a polymer was assembled from the co-polymerisation of nine serologically distinct peptides demonstrated recognition of the polymer by antisera raised to each of the individual epitopes. These findings raise the strong possibility that such polymers could be used diagnostically. The present study also shows that the polymers are strong immunogens and in the case of the polymer containing two different B-cell determinants, antibody was elicited against each one.

A great advantage of the approach described here is that the component synthetic peptide epitopes are purified prior to their incorporation into the immunogen. Furthermore, because the component peptides are individually assembled, other molecular properties such as intra-peptide disulphide loops can be incorporated to mimic similar structures in the native protein. The ability to assemble multiple copies of the same or different peptides using this technology not only provides a way of addressing the polymorphism of some pathogens but also that associated with the MHC antigen system in the recognition of T-cell epitopes. In this way the assembly of appropriate combinations of B- and T-cell epitopes into polymeric supports should allow antibody production in animals of a wide range of different histocompatibility types. This is of great importance in the design of any vaccine to be administered to an outbred population such as man or his domestic animals. If adjuvants are found to be necessary, new generation materials such as PAM₃Cys²⁹ can be assembled into the polymers. The incorporation of hydrophobic sequences into polymeric synthetic peptides will allow them to be anchored into liposomes, ISCOMS^{30,33} or other amphipathic materials, such as DOTAP, which have proven successful in adjuvanting protein antigens.

Co-polymerisation of the acryloyl peptides with other reagents allows separation of the peptide epitopes from each other within the polymer but also separation of the epitope from the backbone allowing steric limitations on the polymerisation process to be minimised. In this study we have used Ahx between the peptide and the acryloyl group to space the peptide from the backbone. The shorter of the two B cell determinant peptides, B₂, showed a slightly reduced ability to interact with MAb when in the polymeric form. This may be an accessibility problem which resulted in poly (T+B₂) being a weaker immunogen than poly (T+B₁) but which potentially could be overcome by using a longer spacer sequence. Acrylamide was used to space the peptides from each other. However, it is possible to substitute acrylamide with amino acids acryloylated at their α-amino groups or with other spacing groups. We have constructed polymers using acryloyl-lysine as a spacer and it would also be possible to use other amino acids for their hydrophilic, polar, charged or even hydrophobic properties to confer the appropriate physical properties on the polymer backbone. Lysine, if acryloylated at both the α- and ε-amino groups, could provide cross-linking which may allow formation of a gel with subsequent slow release properties. In these ways, the distances separating the peptide from the polymer backbone or the separation between peptides on the backbone and the charge characteristics of the polymer can be tailored. These approaches are useful where steric hindrance or adventitious interactions between neighbouring peptides needs to be avoided or where it may be advantageous to increase the opportunity of two or more separate peptides to interact with each other, such as in the formation of coiled-coil structures. Inclusion of other reagents containing a radioactive, fluorescent or chemiluminescent reagent in the polymerisation protocol would provide, in side-chains distinct from the peptides, tracer groups; incorporation of protease resistant or protease susceptible sequences between the peptide epitopes and the backbone may also lead to the controlled release of epitopes.

Section 3

Synthesis of Acryloyl Amino Acids

The amino acids H-lysine(Boc)-OtBu, H-glutamic acid(OtBu)-OtBu and H-serine(tBu)-OtBu (Calbiochem-Novabiochem, New South Wales, Australia) were acryloylated using the following conditions: to 3 mmoles of the amino acid in 10ml DCM containing 7.2 mmoles DIPEA (2.4 mole equivalents), 3.6 mmoles (1.2 mole equivalents) of acryloyl chloride (10 mL DCM) was added dropwise under N_{z} at −10° C. The reaction was maintained at −10° C. for 1 hour with vigorous stirring and a further hour at room temperature. The reaction was monitored by the Kaiser test which indicated that the reaction was complete after 2 hours. The reaction mixture was washed 1 ×water, 1 ×5% aqueous NaHCO₃, 1 ×water, 2×10% aqueous citric acid, 1 ×water and 1 ×brine and then dried overnight with Na₂SO₄. After filtration the DCM was removed by rotary evaporation (<30° C.) to a clear oil (99% yield) and the product was pure by TLC (chloroform:methanol 95:5). The amino acid side chain and carboxyl protecting groups was removed with TFA:water (95:5) cleavage mixture for two hours, under N₂, no light. The TFA was removed under a stream of nitrogen and the product rotary evaporated (<30° C.) to a clear oil. The acryloyl amino acid was dissolved in 6 M guanidine-HCI +2 mM EDTA in 0.5M Tris (GuHCl) and the pH adjusted to 8.3 with 4M Tris. A working stock solution was made containing 0.15 mg/μl of an acryloyl amino acid. Each acryloyl amino acid had the expected mass as indicated by FAB mass spectra.

Polymerisation of Acryloyl Peptides with Acryloyl Amino Acids

Polymerisation was carried out as described above. Acryloyl amino acids literally substituted acrylamide as the co-monomer in the formation of peptide polymers with the molar ratio remaining 50:1 (acryloyl amino acid: acryloyl peptide/s). Amino acid based peptide polymers were worked up and purified as describe above for acrylamide based peptide polymers and lyophilised. Amino acid analysis confirmed the presence of peptide in the polymer and the ratio of peptide to amino acid.

Immunisation Protocols

Female BALB/c mice, 6 to 8 weeks old, were used and were inoculated with at the following doses 5, 0.5, 0.05 nmoles of peptide (contained within the polymer) of either poly(serine) ALN+C10, poly(glutamic acid) ALN+C10 or poly(acrylamide) ALN+C10 emulsified in complete Freund's adjuvant (CFA). Mice received a second dose on day 30. All animals were bled from the retro-orbital plexus on day 30 after the primary peptide polymer inoculation and day 12 after the secondary peptide polymer inoculation, the sera was stored at −20° C. until required.

For the cytotoxic T-cell (CTL) study, BALB/c mice were inoculated s.c. in the hind footpads (50μl per footpad) with a total of 10 μg (7.8 nmoles) of the CTL determinant in monomeric or polymeric form. Mice were inoculated intranasally with infectious influenza virus A/Memphis/1/71 at 1×10^{4.5} pfu in 50 μl.

Antibody Binding Assays

ELISA assays were performed as described above. Antibody titres were expressed as the reciprocal of the antibody dilution giving an absorbance of 0.25, which corresponds to at least five times the background value.

Cell Culture Medium

T-cell medium consisted of RPMI-1640 (CSL, Ltd. Parkville, Australia) supplemented with 10% heat inactivated (56° C., 30min) foetal calf serum(vol/vol), 2mM glutamine, 2mM sodium pyruvate, 0.1 mM 2-mercaptoethanol, 30/μg/ml gentamicin, 100 I.U./ml penicillin and 100μg/ml streptomycin.

T Cell Proliferation Assay

T-cell clone 4.51 was used and was raised in mice immunised with purified hemagglutinin heavy chain (HA,) and proliferates in response to the peptide KYVKQNTLKL derived from HA,. The derivation, properties and maintenance of this clone have been described elsewhere₂₈. Prior to use, the cultured cells were passaged over Isoplaque-Ficoll gradients^{22,23}.

Proliferation assays were set up in 96-well flat-bottom microtitre trays (Nunc, Denmark) containing peptide antigen in monomeric or polymeric form (40μM in the first well) with 1×10⁴ T-cells (clone 4.51) in the presence of 3×10⁵ (100μl) γ-irradiated (2,200 rad, ⁶⁰Co source) normal BALB/c spleen cells, as a source of antigen presenting cell in a total volume of 250μl^{22,23}. Cultures were incubated for 4 days at 37° C. in an atmosphere of 5% CO₂ with 1μCi well⁻¹ ³H-thymidine present during the final 18hr. Cells were then harvested onto glass fibre filters and incorporation of ³H-thymidine was measured on a Hewlett Packard Matrix 9600 direct β-counter.

Cytotoxic T-cell Assay

Secondary effector cells were prepared from (i) popliteal and inguinal lymph node cell suspensions from BALB/c mice inoculated 7 days previously with peptide monomer or polymer (10μg of peptide), (ii) spleen cells, depleted of erythrocytes by treatment with tris-buffered ammonium chloride (ATC; 0.15M NH4Cl in 17 mM tris-HCI at pH 7.4) from mice primed at least 21 days previously with infectious virus. The effector cell populations were incubated (3×10⁷ cells/flask) in 25ml of T-cell medium containing 1×10⁷ virus infected or peptide pulsed spleen cells as a source of antigen presenting cells. The virus infected spleen cells had been preincubated at 37° C. for 30 mins with 1000-5000 HAU of infectious virus in 1 ml of serum free RPMI and washed once prior to use, the peptide pulsed cells were incubated with 100μg of the CTL peptide determinant (monomer) in 1 ml of serum free RPMI at 37° C. for 2 hours. Cultures were incubated for 5 days at 37° C. in an atmosphere of 5% CO₂, after which the cells were washed and used in the CTL assay.

P815 mastocytoma cells were used as target cells in the CTL assay, virus infected and uninfected targets were prepared by incubating 2×10⁶ P815 cells in 500 μl of infectious virus solution (1000-5000 HAU mL⁻¹) or serum free RPMI, respectively at 37° C. After 2 hours the cells washed and resuspended in 200 μl of TCM containing 200 μCi ⁵¹Cr (Amersham, Australia). After 2 hours the cells were washed 3 times and their concentration adjusted to 1×10⁵ cells ml⁻¹. One hundred microlitre aliquots (1×10⁴ cells) of the target cells (infected or uninfected) were added to a 96-well U-bottom tissue culture plates and 100 μl of effector cells at the following effector: target cell ratios; 100:1, 50:1, 25:1, 12:1, 6:1, 3:1, 1.5:1 and 0:1 were added. The cells were gently pelleted by centrifugation 600 ×g, 30 seconds and incubated for 4 hours at 37° C. in an atmosphere of 5% CO₂, after which 100 μl of the supernatant was taken and the amount of radioactivity assessed using γ-counter. The percentage of specific ⁵¹Cr released at each effector: target ratio is given by the equation:

$$100 \times \frac{test counts - spontaneous release}{maximum releasable counts - spontaneous release}$$

Spontaneous ⁵¹Cr release from target cells incubated with medium only ranged from 1-10% of the maximum releasable counts. Data are represented as the mean of the values obtained from triplicate cultures.

Results

Proliferation of T-cell Clone 4.51 to Polymerised PKY Containing Enzymatic Cleavage Sequences

Two enzymatic cleavage sequences were chosen and synthesised onto the N-terminus of peptide PKY which was then acryloylated as previously described. The sequences a tetramer (GLFG) and a pentamer (VYLKY) present the cleavage site of cathepsin B³⁸ and the cleavage motif described by van Noort and van der Drift³⁹. Serine, glutamic acid and acrylamide based polymers were assembled containing PKY either with or without the enzymatic cleavage sequences inserted between the peptide and the acryloyl-Ahx- group. Each of these polymers were used to assess their ability to stimulate clone 4.51 in a proliferation assay.

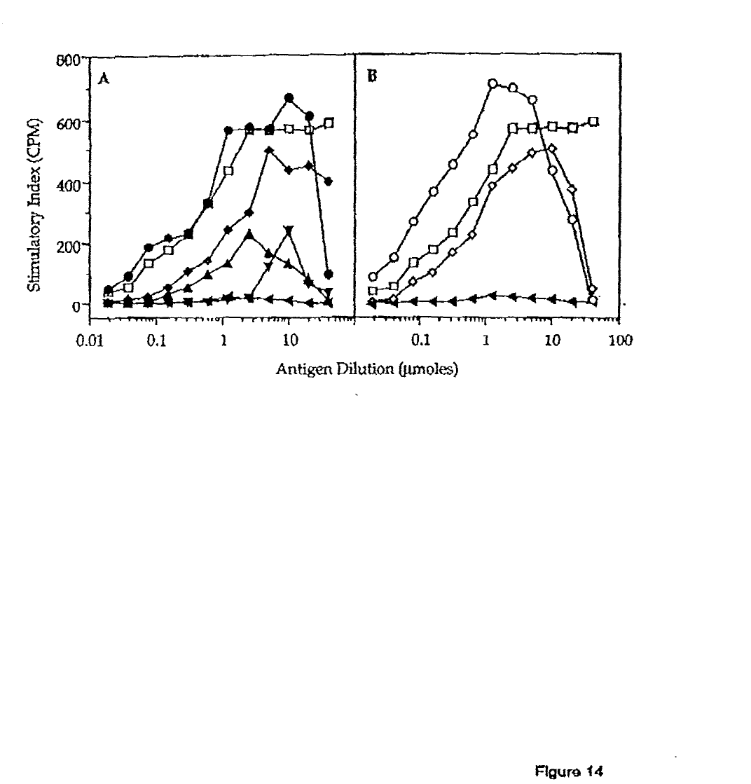

As can be seen in FIG. 14 those polymers which contained either the cathepsin B or the cathepsin D cleavage sequences induced a proliferation response. The incorporation of the cathepsin D sequence induced higher proliferation as compared to the cathepsin B sequence for each type of polymer. Poly(glutamic acid) based polymers elicited a response 35% to 50% higher proliferative response than poly(serine) based polymers at 0.1 μmole dilution. The amino acid based polymer induced a much higher proliferative response than poly(acrylamide) based polymers.

Ability of Peptide Polymers to Induce a Cytotoxic T-lymphocyte Response

The CTL epitope ¹⁴⁷TYQRTRALV¹⁵⁵ from the nucleoprotein of influenza virus was acryloylated and polymerised with acrylamide, acryloyl-serine and acryloyl-glutamic acid. Four lysines were synthesised onto the N-terminus of the GM epitope to act as a spacer between the polymer backbone and the peptide, this peptide was also acryloylated and polymerised with the same co-monomers. The CTL peptide polymers were used to prime BALB/c mice and T-cells were isolated from the inguinal and popliteal lymph nodes after 7 days and were restimulated in vitro with virus or CTL peptide. The restimulated T-cells were then used in a CTL assay to evaluated their ability to kill virus infected or uninfected target cells.

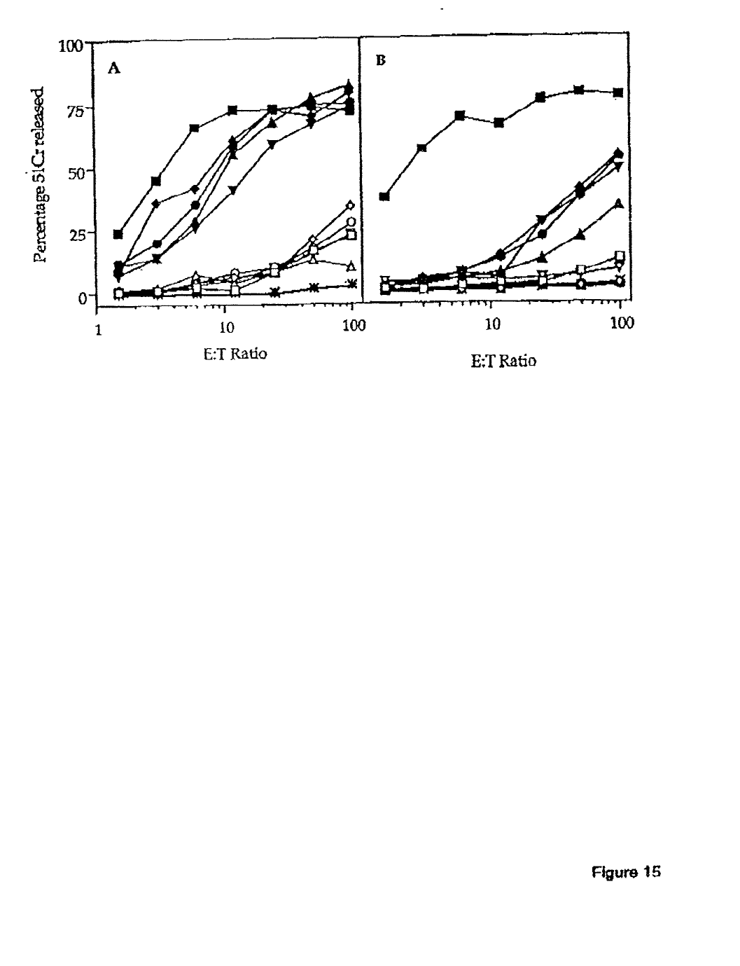

FIG. 15 illustrates that amino acid based polymers containing the CTL or K₄-CTL determinant are able to prime for a CTL response, whereas the CTL peptide polymerised with acrylamide did not. The CTL response induced by the poly(amino acid/s) CTL is comparable to that induced by the CTL peptide alone and similar to that induced by infectious virus (panel A). Although the response induced by restimulating the poly(amino acid) CTL T-cells with CTL peptide is weaker than T-cells derived from mice exposed to infectious virus (panel B) they are still able to kill viral targets. Thus T-cells primed with peptide polymer and then restimulated with peptide are able to kill virus infected targets. A surprising result is that acryloyl-Ahx-CTL did not induce a CTL response whereas CTL monomer and acryloyl-Ahx-K₄CTL did. This may be a reflection of antigen processing events which are able to cleave the acryloyl-Ahx-K₄ from the CTL epitope but not acryloyl-Ahx, which in turn would affect the peptides ability to stimulate a CTL response.

Comparison of Poly(serine)ALN+C10, Poly(glutamic acid) ALN+C10 and poly(acrylamide) ALN+C10 to Elicit Antibodies

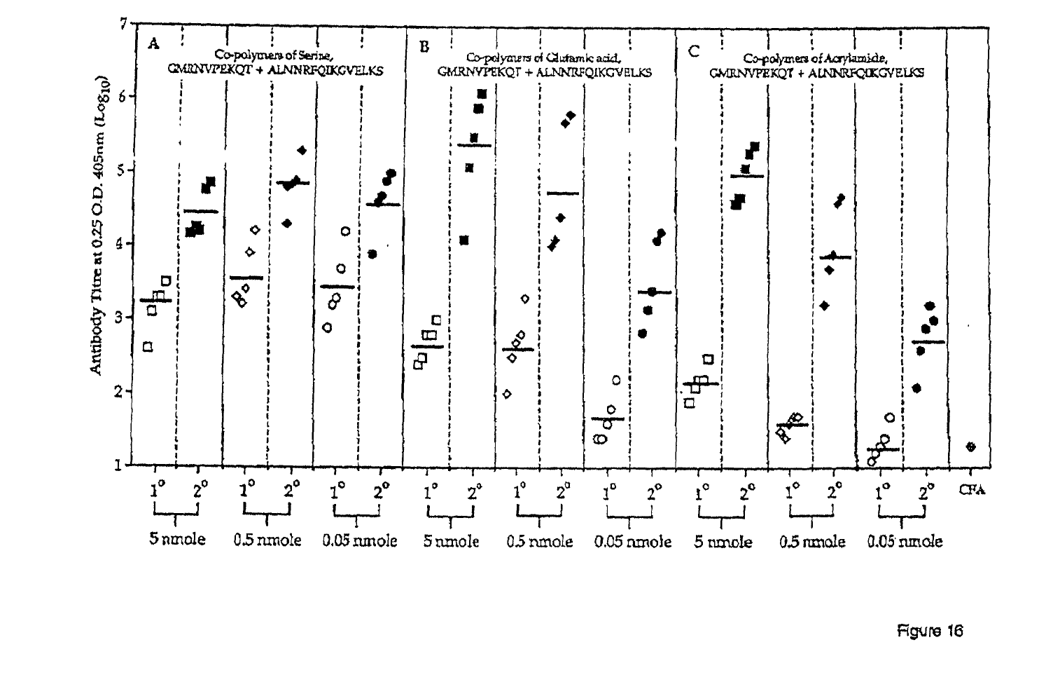

ALN and C10 were polymerised with acryloyl-serine, acryloyl-glutamic acid or acrylamide, emulsified with CFA and inoculated into BALB/c mice at 5, 0.5 and 0.05 nmole peptide doses. The primary and secondary antibody response was determined by ELISA (FIG. 16). All of the polymers were able to induce comparably high antibody titres at 5 and 0.5 nmole peptide dose. Unlike the glutamic acid or acrylamide based polymers which elicited a weaker response at 0.05 nmole dose, serine based polymers elicited an equally high antibody titre.

Section 4

Antigenicity and Immunogenicity of Polymers Synthesised by Polyacryloylation

To evaluate the effect of orientation of a peptide within a polymer, a number of peptide epitopes from the streptococcal M protein were assembled with the acryloyl group at their C-terminus. This was achieved by the addition of Fmoc Lys (Mtt)-COOH (Calbiochem-Novabiochem, New South Wales, Australia) as the C-terminal residue. After the last (N-terminal) amino acid had been added in the synthesis and its Fmoc group removed, the exposed N-terminal amino group was blocked by treating with N-acetyl imidazole or acetic anhydride. Alternatively, the N-terminal amino acid can be added as the BOC-amino acid derivative to allow final exposure of an amino group at the N-terminus if needed but to prevent its acryloylation. The Mtt protecting group at the C-terminus of the peptide was then removed by treating 3 times with a 1% solution of TFA in dichloromethane containing 5% triisopropylsilane for 3 minutes at which point additional spacers can then be added to the amino group exposed by this treatment and the acryloyl group finally attached.

Method

Homopolymers containing multiple copies of one peptide stitched onto the polymeric backbone were synthesised. Homopolymers for the N-terminal peptides ST156(1-19) and M52(1-19) were constructed. For both peptides, a separate construct was made ix which multiple copies of each peptide were attached to the backbone by both the C-terminal end and the N-terminal end. In addition, one heteropolymer containing both the peptides ST156(1-19) and M52(1-19), attached by the C-terminal end onto the same polymeric backbone were constructed.

These polymers were tested for immunogenicity by immunising B10.BR mice. Mice were given an initial immunisation of construct (30 μg/mouse) emulsified in complete Freunds adjuvant (CFA) and boosted with the construct in PBS only at 21, 31, 41 and 51 days. Mice were bled at 9, 19, 29, 49 and 59 days after the initial immunisation. At each time point the sera were tested for peptide-specific antibodies using ELISAs. Anitisera were also tested for opsonic activity using the indirect bactericidal assay.

Results

Homopolymer

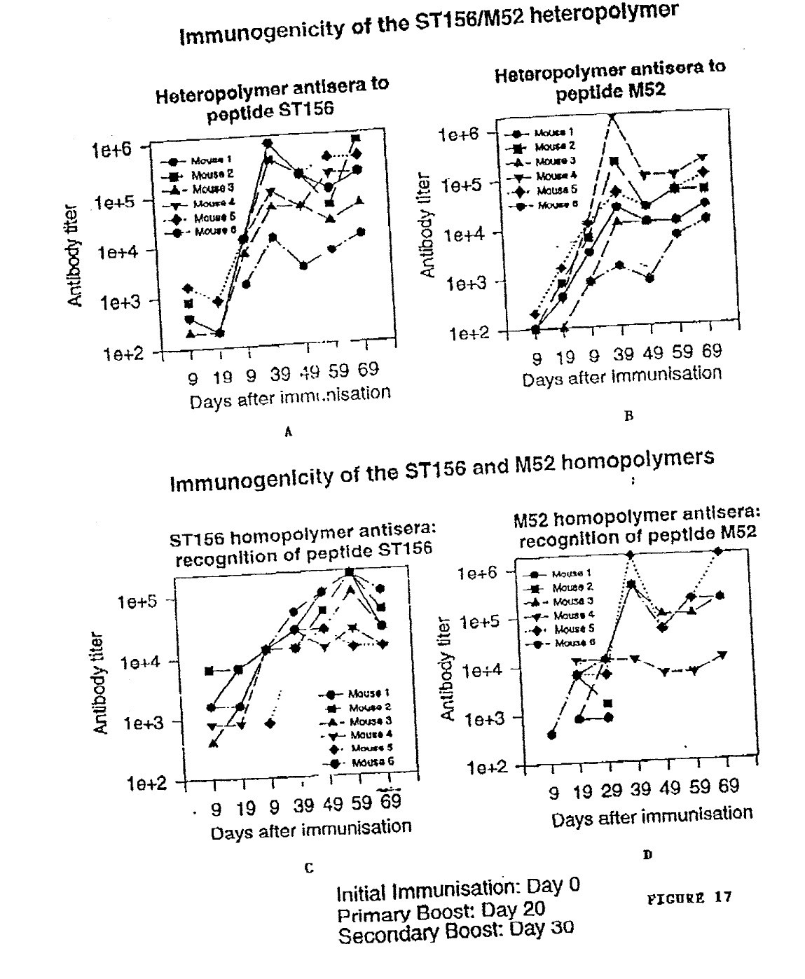

Antisera raised against the ST156(1-19) homopolymer and M52(1-19) homopolymer, with peptides attached by both the N and C-terminal end, were tested in an indirect ELISA to determine whether the peptide antigen from each construct could be recognised. Table 3 summaries the results from sera obtained 59 days after initial immunisation. Antisera from both the ST156(1-19) homopolymer and M52(1-19) homopolymer in which peptides are attached by the N-terminus to the backbone, did not recognise the respective individual peptides with the exception of mouse 2 immunised with the M52(1-19) homopolymer, nor were these sera opsonic for their respective group A streptococci (GAS) strains. However, when peptides are attached by the C-terminal end to the backbone, all mice raised significantly high levels of antibody to their respective peptides (Table 3; FIG. 17).

TABLE 3
Immunogenicity of ST156(1-19) and M52(1-19)-homopolymers

| | Peptide attached to | | Peptide attached to | |
|---|---|---|---|---|
| | backbone by the | | backbone by the | |
| | N-terminal | | C-terminal | |
| Murine antisera | | Titre against | | Titre against |
| raised against the | Mouse | peptide | Mouse | peptide |
| homopolymer | Number | antigen | Number | antigen |
| ST156(1-19) | M1 | 200 | M1 | 204800 |
| Homopolymer | M2 | 200 | M2 | 204800 |
| | M3 | 200 | M3 | 102400 |
| | M4 | 200 | M4 | 25600 |
| | | | M5 | 12800 |
| | | | M6 | 204800 |
| M52(1-19) | M1 | 200 | M1 | 102400 |
| Homopolymer | M2 | >12800 | M2 | 6400 |
| | M3 | 200 | M3 | 204800 |
| | M3 | 200 | M4 | 204800 |

Heteropolymer

Mice were immunised with a M52/ST156 heteropolymer, that is both peptides were attached by the C-terminus onto the same backbone. FIG. 17 shows that sera from mice immunised with the heteropolymer were able to recognise both individual peptides with titres greater than 40000 by 69 days post initial immunisation.

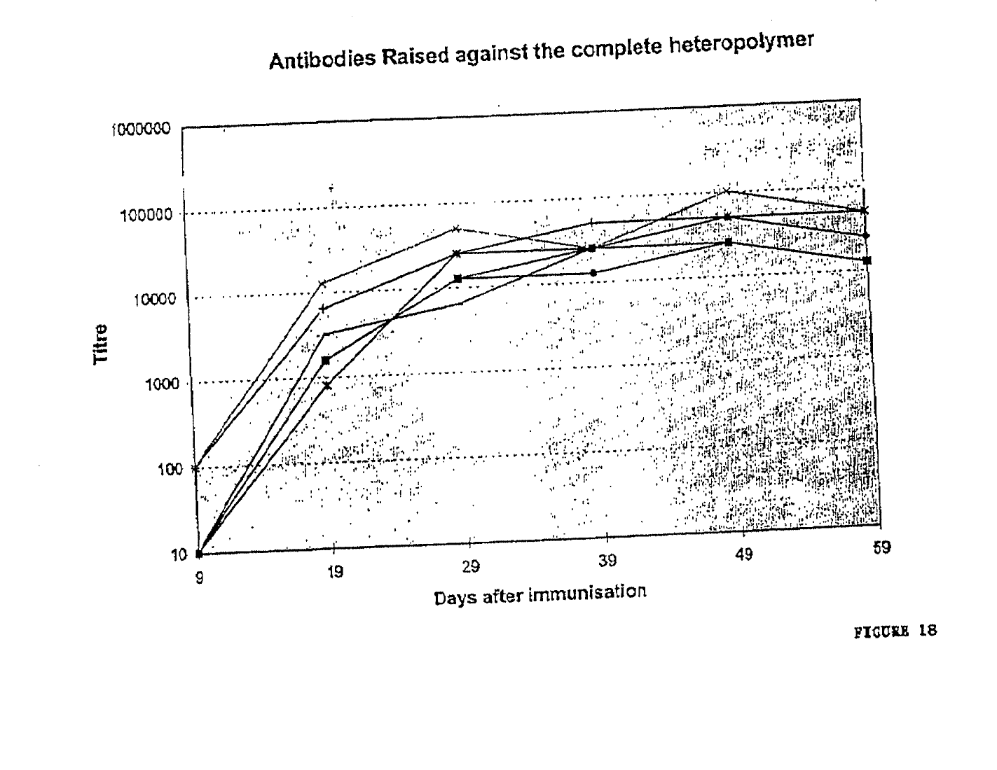

Experiments using a heteropolymer with 8 N-terminal peptides attached by the N-terminal to the backbone showed no significant rise in levels of antibodies to the individual peptides (Table 4). However, significant levels of antibodies were raised to the whole heteropolymer (Table 4; FIG. 18). Competition ELISAs showed that free peptide does not inhibit the antisera raised against the various homopolymers (listed above) and the heteropolymer when the peptides are attached by the N-terminal onto the backbone. Antisera raised specifically to individual peptides alone are reacted back to the heteropolymer in an ELISA, the heteropolymer recognises the peptide specific sera (Table 5), suggesting that the heteropolymer is still antigenic.

TABLE 4
Immunogenicity of antisera raised against an eight peptide
heteropolymer

| Peptide antigen on ELISA plate | Titre against peptide antigen |
|---|---|
| Heterpolymer | 127800-102400 |
| M80(1-19) | 1600 |
| M41(1-19) | 400 |
| M52(1-19) | <100 |
| M55(1-19) | <100 |
| M57(1-19) | <100 |
| NS5(1-19) | <100 |
| NS27(1-19) | <100 |
| ST156(1-19) | <100 |

TABLE 5
Cross-reactivity of peptide-specific antisera to the heteropolymer

| Heteropolymer | | | | | |
|---|---|---|---|---|---|
| on ELISA | Peptide-specific Antisera | | | | |
| plate | M55 | NS1 | ST156 | M52 | Heteropolymer |
| M55 | 6400 | <100 | <100 | <100 | 6400 |
| NS1 | <100 | 6400 | <100 | <100 | 800 |
| ST156 | <100 | <100 | >12800 | 200 | >12800 |
| M52 | <100 | <100 | 400 | >12800 | >12800 |
| M41 | <100 | <100 | <100 | <100 | >12800 |
| M80 | <100 | <100 | <100 | <100 | >12800 |
| NS27 | <100 | <100 | <100 | 3200 | 800 |
| M57 | <100 | <100 | <100 | <100 | <100 |
| Heteropolymer | 800 | <100 | >12800 | >12800 | >12800 |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1 Schutze, M. P., Leclerc, C., Jolivet, M., Audibert, F., and Chedid, L. Carrier-induced epitopic suppression, a major issue for future synthetic vaccines. J Immunol. 1985, 135, 2319-22.

2 Di-John, D., Wasserman, S. S., Torres, J. R., Cortesia, M. J., Murillo, J., Losonsky, G. A, Herrington, D. A, Sturcher, D., and Levine, M. M. Effect of priming with carrier on response to conjugate vaccine. Lancet. 1989, 2, 1415-8.

3 Briand, J. P., Muller, S., and Van-Regenmortel, M. H. Synthetic peptides as antigens: pitfalls of conjugation methods. J Immunol Methods. 1985, 78, 59-69.

4 Togna, A. R., Del-Giudice, G., Verdini, A. S., Bonelli, F., Pessi, A., Engers, H. D., and Corradin, G. Synthetic Plasmodium falciparum circumsporozoite peptides elicit heterogenous L3T4+T cell proliferative responses in H-2b mice. J Immunol. 1986, 137, 2956-60.

5 Grillot, D., Michel, M, Muller, I., Tougne, C., Renia, L., Mazier, D., Corradin, G., Lambert, P. H., Louis, J. A., and Del-Guidice, G. Immune responses to defined epitopes of the circumsporozoite protein of the murine malaria parasite, Plasmodium yoelii. Eur J Immunol. 1990, 20, 1215-22.

6 Grillot, D., Pessi, A., Verdini, A. S., Lambert, P. H., and Del-Giudice, C. Reduced antibody response to the repetitive sequence of the Plasmodium falciparum circumsporozoite protein in mice infected with Plasmodium yoelii blood forms. Med Microbiol Immunol Berl. 1990, 179, 237-44.

7 Leclerc, C., Przewlocki, G., Schutze, M. P., and Chedid, L. A synthetic vaccine constructed by copolymerization of B and T cell determinants. Eur J Inmmunol. 1987, 17, 269-73.

8 Francis, M. J., Hastings, G. Z., Syred, A. D., McGinn, B., Brown, F., and Rowlands, D. J. Non-responsiveness to a foot-and-mouth disease virus peptide overcome by addition of foreign helper T-cell determinants. Nature. 1987, 330, 168-70.

9 Tam, J. P. Synthetic peptide vaccine design: synthesis and properties of a high-density multiple antigenic peptide system. Proc Natl Acad Sci USA. 1988, 85, 5409-13.

10 Tam, J. P., Clavijo, P., Lu, Y. A., Nussenzweig, V., Nussenzweig, R., and Zavala, F. Incorporation of T and B epitopes of the circumsporozoite protein in a chemically defined synthetic vaccine against malaria. J Exp Med. 1990, 171, 299-306.

11 Lu, Y. A., Clavijo, P., Galantino, M., Shen, Z. Y., Liu, W., and Tam, J. P. Chemically unambiguous peptide immunogen: preparation, orientation and antigenicity of purified peptide conjugated to the multiple antigen peptide system. Mol Immunol. 1991, 28, 623-30.

12 Goddard, P., McMurray, J. S., Sheppard, R. C., and Emson, P. A solulisable polymer support suitable for solid phase peptide synthesis and for injection into experimental animals. J. Chem. Soc., Chem. Commun. 1988, 1025-1027.

13 Jackson, D. C., Fitzmaurice, C. J., Sheppard, R. C., McMurray, J., and Brown, L. E. The antigenic and immunogenic properties of synthetic peptide-based T-cell determinant polymers. Biomed Pep Prot and Nucl Acids. 1995, 1, 171-176.

14 Rose, K. Facile synthesis of homogeneous artificial proteins. J Am Chem Soc. 1994, 116, 30-33.

15 Shao, J. and Tam, J. P. Unprotected peptides as building blocks for the synthesis of peptide dendrimers with oxime, hydrazone and thiazolidine linkages. J Am Che Soc. 1995, 117, 3893.

16 Tam, J. P. and Spetzler, J. C. Chemoselective approaches to the preparation of peptide dendrimers and branched artificial proteins using unprotected peptides as building blocks. Biomed Pep Prot and Nucl Acids. 1995, 1, 123-132.

17 Eclkstein, H., Hu, Z., and Schott, H. Synthesis of peptide gels for the investigation of oligopeptide-oligonucleotide interactions. Biopolymers. 1986, 25, 1055-67.

18 Hancock, W. S. and Battersby, J. E. Analyt Biochem. 1976, 71, 261-.

19 Jackson, D. C., Tang, X. L., Brown, L. E., Murray, J. M., White, D. O., and Tregear, G. W. Antigenic determinants of influenza virus hemagglutinin. XII. the epitopes of a synthetic peptide representing the C-terminus of HA1. Virology. 1986, 155, 625-32.

20 Schoofs, P. G., Geysen, H. M., Jackson, D. C., Brown, L. E., Tang, X. L., and White, D. O. Epitopes of an influenza viral peptide recognized by antibody at single amino acid resolution. J Inununol. 1988, 140, 611-6.

21 Jackson, D. C., Drummer, H. E., and Brown, L. E. Conserved determinants for CD4+T cells within the light chain of the H3 hemagglutinin molecule of influenza virus. Virology. 1994, 198, 613-23.

22 Brown, L. E., Ffrench, R. A., Gawler, J. M., Jackson, D. C., Dyall-Smith, M. L., Anders, E. M., Tregear, G. W., Duncan, L., Underwood, P. A., and White, D. O. Distinct epitopes recognized by I-Ad-restricted T-cell clones within antigenic site E on influenza virus hemagglutinin. J Virol. 1988, 62, 305-12.

23 Brown, L. E., Murray, J. M., Anders, E. M., Tang, X. L., White, D. O., Tregear, G. W., and Jackson, D. C. Genetic control and fine specificity of the immune response to a synthetic peptide of influenza virus hemagglutinin. J Virol. 1988, 62, 1746-52.

24 Julius, M. H., Simpson, E., and Herzenberg, L. A. A rapid method for the isolation of functional thymus-derived murine lymphocytes. Eur J Immunol. 1973, 3, 645-649.

25 Jackson, D. C. and Brown, L. E. A synthetic peptide of influenza virus hemagglutinin as a model antigen and immunogen. Pept Res. 1991, 4, 114-24.

26 Fitzmaurice, C. J., Brown, L. E., McInerney, T. L., and Jackson, D. C. The assembly and immunological properties of non-linear synthetic immunogens containing T cell and B cell determinants. Vaccine. 1996, In press,.

27 Ffrench, R. A., Tang, X. L., Anders, E. M., Jackson, D. C., White, D. O., Drummer, H., Wade, J. D., Tregear, G. W., and Brown, L. E. Class II-restricted T-cell clones to a synthetic peptide of influenza virus bemagglutinin differ in their fine specificities and in the ability to respond to virus. J Virol. 1989, 63, 3087-94.

28 Brown, L. E., Jackson, D. C., Tribbick, G., White, D. O., and Geysen, E. M. Extension of a minimal T cell determinant allows relaxation of the requirement for particular residues within the determinant. Int Imrmunol. 1991, 3, 1307-13.

29 Wiesmuller, K. H., Jung, G., and Hess, G. Novel low-molecular-weight synthetic vaccine against foot-and-mouth disease containing a potent B-cell and macrophage activator. Vaccine. 1989, 7, 29-33.

30 Morein, B. and Hoglund, S. Subunit vaccines against infection by enveloped viruses. Adv Biotechnol Processes. 1990, 14, 69-90.

31 Morein, B. Iscoms. Vet Microbiol. 1990, 23, 79-84.

32 Lovgren, K. The serum antibody response distributed in subclasses and isotypes after intranasal and subcutaneous immunization with influenza virus immunostimulating complexes. Scand J Immunol. 1988, 27, 241-5.

33 Lovgren, K. and Morein, B. The requirement of lipids for the formation of immunostimulating complexes (ISCOMS). Biotechnol Appl Biochem. 1988, 10, 161-72.

34 Duncan, R., Rejmanova, P., Kopecek, J. and Lloyd, J. B., Biochimica et Biophysica Aca, 1981, 678,143-150.

35 Spaltenstein, A. and Whitesides, G. M., J. Am. Chem. Soc., 1994, 113, 686-7.

36 Sole, N. A. and Barany, G. J. Org Chem, 1992, 57, 345-47.

37 Pepinsky, R. B., Chen., W. Meier and Wallner., B. P., J. Biol. Chem, 1991, 266, 18244-18249

38 Duncan et al., Mackromol Chem 1983, 184, 1997-2008.

39 van Noort and van der Drift, J Biol. Chem 1989, 264, 14159-14164.

1. A polymer comprising polymerised units of (1) CH₂═CR₄—CO—X—R, and (2) CH₂═CR₃—CO—R₂, and optionally one or more other monomers, in which X is absent or is a spacer having a length equivalent to 1 to 30 single C—C bonds;
R₁ is a peptide, each R₁ being the same or different;
R₂ is NH₂, 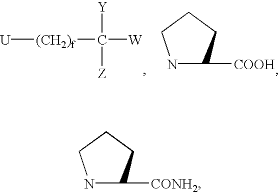
in which U is O or NH;
f is an integer from 0 to 17;
Y is H, COOH, CO—NH₂;
W is H, CH₃, CH₂CO—NH₂, CH₂COOH, CH₂OH, CH(CH₃)OH,
CH₂SH, CH₂CH₂COOH, CH₂CH₂CO—NH₂, CH₂CH(CH₃)₂,
CH(CH₃)CH₂CH₃, (CH₂)₄NH₂, CH₂CH₂SCH3,
CH(CH₃)₂, 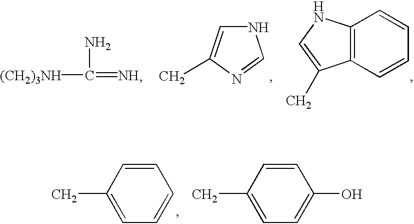
Z is H, (CH₂)ₕQT, in which h is 0-10, Q is O, NH, CH₂ and T is a lipid, labelling molecule, targeting molecule or functional group;
R₃ is CH₃, H, NH₂, OH, CN or halogen, each R₃ being the same or different;
R₄ is CH₃, H, NH₂, OH, CN or halogen, each R₄ being the same or different; and
the ratio of (1):(2) being in the range of about 1:1 to about 1:1000.

2. A polymer as claimed in claim 1 in which there is a plurality of different R₁ groups.

3. A polymer as claimed in claim 2 in which R₁ are epitopes.

4. A polymer as claimed in claim 1 in which the individual R₁ groups are T cell epitopes such that the polymer includes a mixture of T cell epitopes.

5. A polymer as claimed in claim 1 in which the individual R₁ groups are B cell epitopes such that the polymer includes a mixture of B cell epitopes.

6. A polymer as claimed in claim 1 in which the individual R₁ groups are T cell or B cell epitopes such that the polymer includes a mixture of B and T-cell epitopes.

7. A polymer as claimed in claim 1 in which the spacer group X is present such that R₁ is spaced away from the polymer backbone.

8. A polymer as claimed in claim 1 in which X includes an enzymaticly cleavable site.

9. A polymer as claimed in claim 8 in which X includes the amino acid sequence GLFG or VYLKY.

10. A polymer as claimed in claim 1 in which f=0, U=NH₂, Z=H, W=CH₂OH, and Y=COOH or CO—NH₂.

11. A polymer as claimed in claim 1 in which f=0, U=NH₂, Z=H, W=CH₂CH₂COOH, and Y=COOH or CO—NH₂.

12. A polymer as claimed in claim 1 in which the ratio of (1):(2) is in the range of about 1:10 to about 1:50.

13. A polymer formed from CH₂═CR₄—CO—X—R₁ and optionally one or more other monomers, in which X is a spacer having a length equivalent to 1 to 30 single C—C bonds;
R₁ is a peptide, each R₁ being the same or different;
R₄ is CH₃, H, NH₂, OH, CN or halogen, each R₄ being the same or different.

14. A polymer as claimed in claim 13 in which there is a plurality of different R₁ groups.

15. A polymer as claimed in claim 14 in which R₁ are epitopes.

16. A polymer as claimed in claim 13 in which the individual R₁ groups are T cell epitopes such that the polymer includes a mixture of T cell epitopes.

17. A polymer as claimed in claim 13 in which the individual R₁ groups are B cell epitopes such that the polymer includes a mixture of B cell epitopes.

18. A polymer as claimed in claim 13 in which the individual R₁ groups are T cell or B cell epitopes such that the polymer includes a mixture of B and T-cell epitopes.

19. A polymer as claimed in claim 13 in which X includes an enzymaticly cleavable site.

20. A polymer as claimed in claim 8 in which X includes the amino acid sequence GLFG or VYLKY.

21. A polymer as claimed in claim I or claim 13 in which the polymer is cross-linked.

22. A method of preparing a peptide polymer having a polymer backbone and a plurality of pendant peptides attached thereto, the method comprising the steps of:
(i) preparing monomers (1) CH₂═CR₄—CO—X—R₁ and (2) CH₂═CR₃—CO—R₂, and optionally one or more other monomers, in which X is absent or is a spacer having a length equivalent to 1 to 30 single C—C bonds;
R₁ is a peptide, each R₁ being the same or different;
R₂ is NH₂, 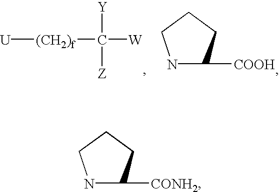
in which U is O or NH;
f is an integer from 0 to 17;
Y is H, COOH, CO—NH₂;
W is H, CH₃, CH₂CO—NH₂, CH₂COOH, CH₂OH, CH(CH₃)OH,
CH₂SH, CH₂CH₂COOH, CH_{z}CH₂CO—NH₂, CH₂CH(CH₃)₂,
CH(CH₃)CH₂CH₃, (CH₂)₄NH₂, CH₂CH₂SCH₃,
CH(CH₃)₂, 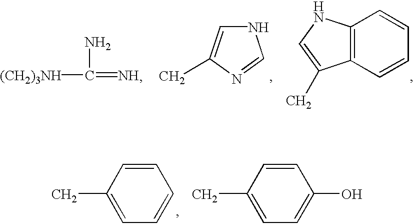
Z is H, (CH₂)ₕQT, in which h is 0-10, Q is O, NH, CH₂ and T is a lipid, labelling molecule, targeting molecule or functional group;
R₃ is CH₃, H, NH₂, OH, CN or halogen, each R₃ being the same or different;
R₄ is CH₃, H, NH₂, OH, CN or halogen, each R₄ being the same or different; and
(ii) polymerising the monomers in the presence of a free radical initiator.

23. A method of raising an immune response in an animal to peptide epitope, the method comprising administering to the animal a composition comprising an acceptable carrier and a polymer comprising polymerised units of (1) CH₂═CR₄—CO—X—R₁ and (2) CH₂═CR₃ —CO—R₂, and optionally one or more other monomers, in which X is absent or is a spacer having a length equivalent to 1 to 30 single C—C bonds;
R₁ is a peptide, each R₁ being the same or different, at least one of R₁being the peptide epitope;
R₂ is NH₂, 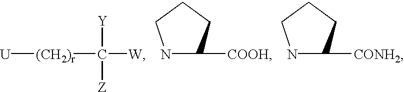
in which U is O or NH;
f is an integer from 0 to 17;
Y is H, COOH, CO—NH₂;
W is H, CH₃, CH₂CO—NH₂, CH₂COOH, CH₂OH, CH(CH₃)OH,
CH₂SH, CH₂CH₂COOH, CH₂CH₂CO-NH₂, CH₂CH(CH₃)₂,
CH(CH₃)CH₂CH₃, (CH₂)₄NH₂, CH₂CH₂SCH₃,
CH(CH₃)₂, 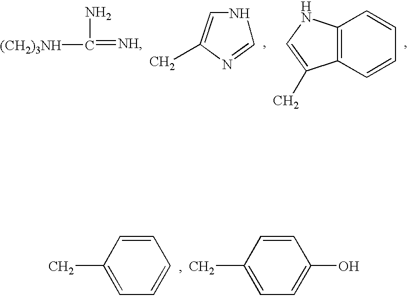
Z is H, (CH₂)ₕQT, in which h is 0-10, Q is O, NH, CH₂ and T is a lipid, labelling molecule, targeting molecule or functional group;
R₃ is CH₃, H, NH₂, OH, CN or halogen, each R₃ being the same or different;
R₄ is CH₃, H, NH₂, OH, GN or halogen, each R₄ being the same or different; and
the ratio of (1):(2) being in the range of about 1:1 to about 1:1000.

24. A method as claimed in claim 23 in which the spacer group X is present such that R₁ is spaced away from the polymer backbone.

25. A method as claimed in claim 23 in which X includes an enzymaticly cleavable site.

26. A method as claimed in claim 25 in which X includes the amino acid sequence GLFG or VYLKY.

27. A method as claimed in claim 23 in which f=0, U=NH₂, Z=H, W=CH₂OH, and Y=COOH or CO—NH₂.

28. A method as claimed in claim 23 in which f=0, U=NH_{Z}, Z=H, W=CH₂CH₂COOH, and Y=COOH or CO—NH₂.

29. A method as claimed in claim 23 in which the ratio of (1):(2) is in the range of about 1:10 to about 1:50.

30. A method as claimed in claim 23 in which the polymer is cross-linked.


SEQUENCE LISTING (28 sequences)

SEQ ID NO: 1 - protein, 4 aa, Artificial Sequence
  Description of Artificial Sequence  Synthetic Peptides
GLFG

SEQ ID NO: 2 - protein, 5 aa, Artificial Sequence
  Description of Artificial Sequence  Synthetic Peptides
VYLKY

SEQ ID NO: 3 - protein, 23 aa, Artificial Sequence
  Description of Artificial Sequence  Synthetic Peptides
PKYVKQNTLKLATGMRNVPEKQT

SEQ ID NO: 4 - protein, 12 aa, Artificial Sequence
  Description of Artificial Sequence  Synthetic Peptides
PKYVKQNTLKLA

SEQ ID NO: 5 - protein, 17 aa, Artificial Sequence
  Description of Artificial Sequence  Synthetic Peptides
VYLKYPKYVKQNTLKLA

SEQ ID NO: 6 - protein, 16 aa, Artificial Sequence
  Description of Artificial Sequence  Synthetic Peptides
GFLGPKYVKQNTLKLA

SEQ ID NO: 7 - protein, 9 aa, Artificial Sequence
  Description of Artificial Sequence  Synthetic Peptides
TYQRTRALV

SEQ ID NO: 8 - protein, 12 aa, Artificial Sequence
  Description of Artificial Sequence  Synthetic Peptides
KKKTYQRTRALV

SEQ ID NO: 9 - protein, 10 aa, Artificial Sequence
  Description of Artificial Sequence  Synthetic Peptides
GMRNVPEKQT

SEQ ID NO: 10 - protein, 15 aa, Artificial Sequence
  Description of Artificial Sequence  Synthetic Peptides
ALNNRFQIKGVELKS

SEQ ID NO: 11 - protein, 24 aa, Artificial Sequence
  Description of Artificial Sequence  Synthetic Peptides
CPKYVKQNTLKLATGMRNVPEKQT

SEQ ID NO: 12 - protein, 8 aa, Artificial Sequence
  Description of Artificial Sequence  Synthetic Peptides
RNVPEKQT

SEQ ID NO: 13 - protein, 7 aa, Artificial Sequence
  Description of Artificial Sequence  Synthetic Peptides
TLKLATG

SEQ ID NO: 14 - protein, 11 aa, Artificial Sequence
  Description of Artificial Sequence  Synthetic Peptides
GMRNVPERKQT

SEQ ID NO: 15 - protein, 14 aa, Artificial Sequence
  Description of Artificial Sequence  Synthetic Peptides
PKYVKQNTLKLATG

SEQ ID NO: 16 - protein, 14 aa, Artificial Sequence
  Description of Artificial Sequence  Synthetic Peptides
DCTLIDDALLGDPH

SEQ ID NO: 17 - protein, 11 aa, Artificial Sequence
  Description of Artificial Sequence  Synthetic Peptides
SQHWSYGLRPG

SEQ ID NO: 18 - protein, 20 aa, Artificial Sequence
  Description of Artificial Sequence  Synthetic Peptides
DRAAGQPAGDRAAGQPAGDR

SEQ ID NO: 19 - protein, 4 aa, Artificial Sequence
  Description of Artificial Sequence  Synthetic Peptides
NANP

SEQ ID NO: 20 - protein, 20 aa, Artificial Sequence
  Description of Artificial Sequence  Synthetic Peptides
FNNTVSFWLRVPKVSASHLE

SEQ ID NO: 21 - protein, 14 aa, Artificial Sequence
  Description of Artificial Sequence  Synthetic Peptides
QYIKANSFIGITEL

SEQ ID NO: 22 - protein, 20 aa, Artificial Sequence
  Description of Artificial Sequence  Synthetic Peptides
LRRDLDASREAKKQVEKALE

SEQ ID NO: 23 - protein, 20 aa, Artificial Sequence
  Description of Artificial Sequence  Synthetic Peptides
YIYADGKMVNEALVRQGLAK

SEQ ID NO: 24 - protein, 12 aa, Artificial Sequence
  Description of Artificial Sequence  Synthetic Peptides
DLIAYLKQATAK

SEQ ID NO: 25 - protein, 4 aa, Artificial Sequence
  Description of Artificial Sequence  Synthetic Peptides
GFGA

SEQ ID NO: 26 - protein, 5 aa, Artificial Sequence
  Description of Artificial Sequence  Synthetic Peptides
LKLAT

SEQ ID NO: 27 - protein, 10 aa, Artificial Sequence
  Description of Artificial Sequence  Synthetic Peptides
KYVKQNTLKL

SEQ ID NO: 28 - protein, 16 aa, Artificial Sequence
  Description of Artificial Sequence  Synthetic Peptides
ALNNRNFQIKGVELKS